(19) United States
(12) Patent Application Publication
(10) Pub. No.: US 2010/0074906 A1
(43) Pub. Date: Mar. 25, 2010

(54) Antibodies Specific for Varicella Zoster Virus

(75) Inventors: Roberto Burioni, Rimini (IT); Massimo Clementi, Iesi (IT)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: RIBOVAX BIOTECHNOLOGIES SA, Petit-Lancy (CH)

(21) Appl. No.: 12/525,866

(22) PCT Filed: Feb. 6, 2008
(86) PCT No.: PCT/IB2008/000266

§ 371 Date: Nov. 24, 2009

(30) Foreign Application Priority Data
Feb. 6, 2007 (EP) 07002478.1

(51) Int. Cl. A61K 39/42; C07K 14/435; A61P 31/12; C07K 16/00; C07K 16/08; C07H 21/04; C12N 15/63; C12N 5/10; C12N 1/00; C12P 21/02; C12Q 1/70

(52) U.S. Cl. 424/159.1; 530/325; 530/350; 530/387.1; 530/389.4; 536/23.53; 435/320.1; 435/325; 435/243; 435/69.1; 435/5

(57) ABSTRACT

The present invention provides novel antibody sequences that bind Varicella Zoster Virus (VZV) and neutralize VZV infection. The novel sequences can be used for the medical management of VZV infection, in particular for detecting the virus or for preparing pharmaceutical compositions to be used in the prophylactic or therapeutic treatment of VZV infection.

### FIELD OF THE INVENTION

The invention relates to novel antibody sequences isolated from phage display libraries having biological activities specific for a virus.

### BACKGROUND OF THE INVENTION

Phage display technologies take advantage of the small dimension and the adaptability of the genome of filamentous phage (such as M13) that infect bacterial cells (e.g. Escherichia coli cells) for cloning, selecting, and engineering polypeptides (antibody fragments, bioactive peptides, enzymes, etc.) that are expressed on their surface and can exert biological functions following their interaction with a target.

Several cloning and expression strategies, vectors, libraries, methods for propagating phage, and screening assays have been developed for different applications, as reviewed in articles (Bradbury A and Marks J, 2004; Mancini et al., 2004; Conrad U and Scheller J, 2005; Hust M and Dubel S, 2005), and books (“Phage display: A Practical Approach”, vol. 266, ed. Clackson and Lowman H, Oxford Univ. Press, 2004; “Phage Display: A Laboratory Manual”, ed. Burton D et al., CSHL Press, 2001).

Phage display libraries are made of a population of recombinant phage, each presenting a single element of a repertoire of protein sequences. Phage that express specific proteins can be isolated from the library by iterative affinity-based and/or function-based selection processes (the “panning”). For example, the proteins can be antibody fragments, in the form of variable heavy/light chain heterodimers (commonly named as Fabs) or single chain Fragment variable (scFv), that can be isolated and characterized on the basis of their affinity for purified antigens or of activity in biological assays.

In particular, screening processes have been developed to identify antibody fragments that have high affinity and specificity for pathogens and biological targets, sometimes with relevant biological activity associated to such binding properties. In fact, an entire therapeutic approach (named passive immunotherapy or passive serotherapy) has been built on the antigen-binding features of antibodies and antibody fragments directed against human or non-human therapeutic targets (Dunman P and Nesin M, 2003; Keller M and Stiehm E, 2000). Passive immunotherapy consists of the administration to individuals of pharmaceutical compositions comprising therapeutic antibodies with a defined binding specificity for a pathogenic antigen (a toxin, a human protein, a virus, or a parasite, for example).

Passive immunotherapy has been introduced into clinical practice, rapidly expanding the opportunities for the treatment of a wide variety of diseases (including infectious diseases, immune-mediated diseases and cancer). This approach can be particularly effective in patients whose immune system is unable to produce them in the amounts and/or with the specificity that are required to block and/or eliminate the targeted molecule (Chatenoud L, 2005; Laffly E and Sodoyer R, 2005).

Among pathogenic antigens that can be targeted using therapeutic antibodies, viruses that infect human cells are of particular importance. The administration of such antibodies can inhibit the propagation of the virus in the patient, and potentially block the outbreak of a viral infection in the population. Alternatively, the antibody may be administered to a patient having a weakened immune system for a more or less prolonged period of time (e.g. immunosuppressed, elderly, or transplanted individuals) that become much more sensitive to infectious diseases, including those that normally do have not serious and/or permanent consequences on health of immunocompetent individuals.

Varicella Zoster Virus (VZV, chickenpox) is one of the viruses that is responsible of such potentially life-threatening infection in immunocompromised individuals. VZV is an alphaherpesvirus encoding at least 70 distinct open reading frames (ORFs), most of them closely related to Herpes Simplex Virus (Mori I and Nishiyama Y, 2005). VZV is transmitted in the human population by direct contact with infectious virus in skin lesions or in respiratory secretion, showing a specific tropism for skin and T cells. Primary VZV infection is directed to lymphocytes followed with a cell-associated viremia, viral replication in organs, and a diffuse cutaneous rash due to secondary viremia. VZV infection can be spread by the release of virions or by cell-to-cell transmission, and establishes latency in sensory ganglia (Arvin A, 1996; Quinlivan M and Breuer J, 2006).

A single VZV attack or vaccination usually confers lifelong protection. However, VZV has intrinsic properties that allow the virus to evade both innate and acquired immune responses. VZV re-infection or re-activation is still possible, also because VZV-specific antibodies may be lost despite detectable cell mediated immunity after vaccination (Ludwig B et al., 2006).

The mechanisms and the potency of the immune response to infection and vaccination have been studied in humans and in animal models (Maple P et al., 2006; Matsuo K et al., 2003; Kutinova L et al., 2001; Massaer M et al., 1999; Haumont M et al., 1997; Hasnie F et al., 2007) or in vitro culture systems (Finnen R et al., 2006; Andrei G et al., 2005a). VZV pathogenesis and immunobiology can be studied in transgenic mouse models, using ex vivo human skin or ganglia models (Baiker A et al., 2004; Ku C et al., 2005; Taylor S and Moffat J, 2005; Zerboni L et al., 2005). In this manner, cell-type-specific VZV apoptotic activities (Hood C et al., 2003) and cellular/viral mediators of VZV infection (Berarducci B et al., 2006; Chen J et al., 2004; Li Q et al., 2007; Hambleton S et al., 2007) have been identified.

A live attenuated varicella vaccine (Oka/Merck strain) is available and it recommended for routine childhood immunization and in adults, given its safety and efficacy (Oxman M et al., 2006; Arvin A, 1996). Even though VZV vaccination is widely established in the industrialized world and is highly effective in reducing all forms of Varicella (especially severe disease) in the short/medium term, the pool of latent or evolving wild-type virus in the population represents a continuing threat (Hambleton S and Gershon A, 2005).

VZV strains have been associated to specific genetic mutations in VZV transcripts (Grose C, 2006) and to three major distinct genotypes (Norberg P et al., 2006). Genetic variants were associated to rash-forming VZV genotypes that were detected within immunized human hosts (Quinlivan M et al., 2007). Moreover, the level of VZV immunity varies considerably in different human populations, for example within the European regions (Nardone A et al., 2007).

Herpes zoster (or shingles) is still observed frequently in clinical practice, especially in the elderly as a result of aging-related waning of cell mediated immunity or due to risk factors for VZV re-activation in the different age spectrums and with concomitant immunodeficiencies due to cancer, immunosuppressing treatments, or steroid therapy. Complications of Herpes Zoster can be neurologic (postherpetic neuralgia, cerebral vasculitis, encephalitis, aseptic meningitis, cranial palsy, or meningoencephalitis), ocular (Herpes Zoster Ophthalmicus, uveitis, retinal necrosis, optic neuritis, or keratitis) or visceral (hepatitis, pneumonitis, myocarditis). The risk factors for Herpes Zoster are becoming better understood but their increasing number and VZV persistence in sensory ganglia and ocular surface suggest the need of a more widespread vaccination in adults and more efficient anti-VZV treatments (Dworkin R et al., 2007; Weinberg J, 2007; Liesegang T, 2004).

VZV infection is extremely dangerous in immunosuppressed patients, in whom the use of vaccines is not advisable. In fact, VZV is a great problem in transplant hospital wards where a single VZV re-activation or re-infection in a single individual in an immunosuppressed state can infect several patients, so that VZV may invade several tissues, including the spinal cord or cerebral arteries (Chaves Tdo S. et al., 2005; Gilden D, 2004). In pregnancy, VZV infection can also spread to the fetus by intrauterine transmission or neonatal infection, causing intrauterine death or congenital varicella syndrome, with serious skin lesions and defects in limb and organ development (Schleiss M. 2003; Sauerbrei A and Wutzler P, 2007).

Apart from vaccination, which has some important contraindications (Arvin A, 1996), antiviral treatments are available (such as acyclovir, valaciclovir, famciclovir, and brivudin). However, in addition to the problem of drug-resistant VZV strains, these compounds may also have contraindications, for example due to the interaction with human enzymes (Andrei G et al., 2005b; Abdel-Haq N et al. 2006). Other treatments such as corticosteroids or anticonvulsants are used for providing additional pain relief but their adverse effect profile limits use, especially for post-herpetic neuralgia (Tyring S, 2007) Thus, alternative approaches are needed when present means for prophylaxis are either not applicable or no longer effective (Hambleton S and Gershon A, 2005). In view of vaccine failure and waning vaccine-induced immunity, second vaccine dose is now recommended (Chaves S et al., 2007).

In order to identify VZV infections early, several diagnostic tests have been developed for detecting VZV-specific antibodies, antigens, or transcripts in similar population of patients, as well as in elderly or immunocompromised individuals being exposed to the risk of VZV infection (Hambleton S and Gershon A, 2005; Smith J et al., 2001).

VZV-specific immune activities were identified in serum, cerebrospinal fluid or oral fluids in connection to VZV-related ocular infections (Kezuka, T. 2004), vasculopathy (Burgoon M et al., 2003), or epidemiological studies (Talukder Y et al., 2005). VZV post-exposure prophylaxis has been proposed and tested, in particular using purified preparations of human antibodies having significant anti-VZV titers that can be administered to prevent the infection (Keller M and Stiehm E, 2000). Specific intravenously injectable preparations of human immunoglobulin having a high titer of anti-VZV antibody have been described (U.S. Pat. No. 4,717,564; CDC, 2006). Their use, alone or in combination with other compounds as VZV-specific antivirals, has been tested in transplanted patients, pregnant women, or newborn infants (Huang Y et al., 2001; Carby Metal., 2007; Koren G et al., 2002).

VZV-specific antigens, variants of VZV glycoproteins, and their immunogenic epitopes, have been described using different antibody preparations, such as human sera (U.S. Pat. No. 6,960,442; Fowler W et al., 1995; Kjartansdottir A et al., 1996; WO 96/01900), non-human polyclonal sera (U.S. Pat. No. 5,306,635; WO 92/06989). Monoclonal antibodies against VZV were isolated and expressed using trioma or hybridomas of human (WO 86/02092; WO 91/16448; WO 95/04080; EP148644; Nemeckova S et al., 1996; Foung S et al., 1985; Sugano T et al., 1987; Yokoyama T et al., 2001; Ito M et al., 1993) or murine origin (EP321249; Vafai A and Yang W, 1991; Montalvo E and Grose C 1986; Forghani B et al., 1994; Grose C et al., 1983; Lloyd-Evans P and Gilmour J, 2000; Shankar V et al., 2005; Garcia-Valcarcel M et al., 1997). Recombinant, VZV-binding and/or neutralizing antibody fragments have been identified in phage display libraries and expressed as single-chain variable fragments (Kausmally L et al., 2004; Drew P et al., 2001) or Fabs (Suzuki K et al., 2007; Williamson R et al., 1993). Anti-VZV murine monoclonal antibodies have been humanized (WO 95/31546).

Although both vaccine and systemic antivirals have brought major improvements, the disease persists. Therapy lessens but does not eliminate many of VZV complications that may manifest in unpredictable patterns. The identification and the production of novel antibodies and antibody fragments that can more efficiently detect and block VZV infection and propagation in the population is still of particular importance for establishing improved treatments for the therapy and/or the prevention of this infectious disease.

### SUMMARY OF THE INVENTION

The present invention provides novel antibody sequences that bind and neutralize VZV, and that can be used for detecting, treating, inhibiting, preventing, and/or ameliorating VZV infection or a VZV-related disease. A panel of human antibody sequences were displayed on recombinant phage and VZV-specific binding activities have been detected in the phage library. The DNA sequences that encode the heavy and light chain variable regions of two antibody fragments and that have VZV-neutralizing activity were identified and named as DDF-VZV1 and DDF-VZV2. The corresponding protein sequences and the Complementarity Determining Regions (CDRs) that are responsible for the VZV-specific biological activity were determined.

The sequences of the invention can be used for producing recombinant proteins having VZV-specific binding and neutralizing properties, in the form of full antibodies, antibody fragments, or any other format of functional protein (in particular fusion proteins) using appropriate technologies for producing recombinant proteins.

Compositions having therapeutic, prophylactic, and/or diagnostic utility in the management of VZV infection and VZV-related diseases can be prepared using the proteins of the Invention. Such compositions may be used to supplement or replace present VZV treatments based on antiviral compounds and/or intravenous immunoglobulin (IVIg) preparations.

Further embodiments of the present invention, including isolated DNA and protein sequences, vectors, recombinant phage, and host cells as well as medical methods and uses, are provided in the following description.

### DESCRIPTION OF THE FIGURES

FIG. 1: Specificity of the VZV binding activity for preparations of recombinant phage expressing DDF-VZV1 (VZV-1), DDF-VZV2 (VZV-2), or an unrelated human Fab (e-137) that was used as a negative control. The binding activity was measured in ELISA using the indicated antigens that were used for plate coating in the form of total protein extracts (for MRC-5 cells) or purified protein (for Bovine Serum Albumin, BSA).

FIG. 2: (A) Alignment of the DNA (lower case) and protein (upper case) sequence of the variable region for heavy chain of DDF-VZV1 (DDF-VZV1 VH; SEQ ID NO.: 1 and 2). The predicted CDRs (HCDR1, HCDR2, and HCDR3; SEQ ID NO.: 3, 4, and 5) are underlined. (B) Alignment of the DNA (lower case) and protein (upper case) sequence of the variable region for the light chain of DDF-VZV1 (DDF-VZV1 VL; SEQ ID NO.: 6 and 7). The predicted CDRs (LCDR1, LCDR2, and LCDR3) are underlined.

FIG. 3: (A) Alignment of the DNA (lower case) and protein (upper case) sequence of the variable region for the heavy chain of DDF-VZV2 (DDF-VZV2 VH; SEQ ID NO.: 8 and 9). The predicted CDRs (HCDR1, HCDR2, HCDR3; SEQ ID NO.: 10, 11, and 12) are underlined. (B) Alignment of the DNA (lower case) and protein (upper case) sequence of the variable region for the light chain DDF-VZV2 (DDF-VZV2 VL; SEQ ID NO.: 13 and 14). The predicted CDRs (LCDR1, LCDR2, LCDR3) are underlined.

FIG. 4: Immunofluorescence of VZV-infected MRC-5 cells and stained with DDF-VZV1 (A) and DDF-VZV2 (B), The cell membrane is indicated with a white line and nuclear membrane is indicated with a dotted white line. No staining was obtained using these Fabs on uninfected cells.

FIG. 5: VZV neutralization activity for DDF-VZV1 (A) and DDF-VZV2 (B), as expressed as partially purified human recombinant Fabs displayed on phage cost proteins. The dose-response analysis of plaque reduction was performed in parallel with a corresponding concentration of an unrelated human Fab (e137). The percentage values were calculated by comparing the data obtained using VZV pre-incubated without any Fab.

FIG. 6: (A) Forward linker for FLAGhis tag (pDD-FLAGhis forward: SEQ ID NO: 15) with the indication of the protein tags (FLAGHis tag; SEQ ID NO: 17) that are consecutively encoded by this oligonucleotide after that is paired to the corresponding reverse primer (pDD-FLAGhis reverse: SEQ ID NO: 16), digested with SpeI and NheI, and cloned in a pDD vector for substituting the cp3* sequence (WO 07/007,154). (B) Schematic map of DNA fragment that contain a heavy chain (HC) and the light chain (LC) of an antibody fragment that is cloned in a pDLacI-FLAGhis vector. The HC sequence is cloned in frame between a PelB signal sequence (PelB) and the FLAGhis tag sequence (FLAGhis). The LC sequence is cloned in frame with a PelB signal sequence (PelB) and its expression is driven by a LacZ pnnoter (LacZ). Between the two expression units, the marker gene (Zeocin; Zeo gene) and the gene controlling LacZ promoter (Lac I gene) are cloned with their own promoters. The sequence coding for cp8* (cp8*) is not transcribed and translated in absence of a functional promoter and start of translation. The relevant restriction sites, in particular those used for cloning HC (SpeI and XhoI), LC (XbaI and SacI), and Lad gene (Stul) are indicated. The corresponding NheI-BglI fragment for expressing DDF-VZV1 using the pDLac system is provided as SEQ ID NO.:18. (C) Coomassie staining of DDF-VZV1-FLAGhis that was expressed using pDLac-VZV1-FLAGhis purification. This figure shows five consecutive fractions eluted from the affinity chromatography column. Fractions were pooled together, concentrated and stored at −20° C. for further use.

FIG. 7: (A) Protein sequence for the heavy chain of human Fab DDF-VZV1, as expressed using pDLac-VZV1-FLAGhis (DDF-VZV1 CHTag; SEQ ID NO.: 20). The corresponding DNA sequence is provided as SEQ ID NO.: 19. The variable region of this heavy chain that was originally cloned using a pDD vector (SEQ ID NO.: 2) is underlined. The PelB sequence is comprised between amino acids 1 and 26. Amino acids 155-260 correspond to amino acids 1-106 of human Ig gamma-1 chain C region (SwissProt Acc. No.: P01857). Amino acids 262-275 correspond to the FLAGhis sequence. (B) Protein sequence for the light chain of human Fab DDF-VZV1, as expressed using pDLac-VZV1-FLAGhis (DDF-VZV1 CL; SEQ ID NO.: 22). The corresponding DNA sequence is provided as SEQ ID NO.: 19. The variable region of this light chain that was originally cloned using a pDD vector (SEQ ID NO.: 7) is underlined. The PelB sequence is comprised between amino acid 1 and 22. Amino acids 129-234 correspond to amino acids 1-106 of Ig kappa chain C region (SwissProt Acc. No.: P01834). (C) Protein sequence for the heavy chain of human Fab DDF-VZV2, as expressed using pDLac-VZV2-FLAGhis (DDF-VZV2 CHTag; SEQ ID NO.: 24). The corresponding DNA sequence is provided as SEQ ID NO.: 23. The variable region of this heavy chain that was originally cloned using a pDD vector (SEQ ID NO.: 9) is underlined. The PelB sequence is comprised between amino acids 1 and 26. Amino acids 152-257 correspond to amino acids 1-106 of human Ig gamma-1 chain C region (SwissProt Acc. No.: P01857). Amino acids 259-272 correspond to the FLAGhis sequence. (D) Protein sequence for the light chain of human Fab DDF-VZV2, as expressed using pDLac-VZV2-FLAGhis (DDF-VZV2 CL; SEQ ID NO.: 26). The corresponding DNA sequence is provided as SEQ ID NO.: 25. The variable region of this light chain that was originally cloned using a pDD vector (SEQ ID NO.: 14) is underlined. The PelB sequence is comprised between amino acid 1 and 22. Amino acids 135-240 correspond to amino acids 1-106 of Ig kappa chain C region (SwissProt Acc. No.: P01834).

FIG. 8: VZV neutralization activity for human Fabs DDF-VZV1 and DDF-VZV2 as expressed and purified using the pDLac system. (A) Each Fab was used at the indicated concentration and the plaque reduction was calculated. (B) Each Fab was used at the indicated concentration either alone or a Fab preparation comprising each Fab in equal amount. The dose-response analysis of plaque reduction was performed in parallel with a corresponding concentration of an unrelated human Fab produced using the same system (e137). The percentage values were calculated by comparing the data obtained using VZV pre-incubated without any Fab (negative control).

### DETAILED DESCRIPTION OF THE INVENTION

The pDD phagemid and the related methods described in WO 07/007,154 allow the cloning, the expression, and the selection of protein sequences that are fused to either one or the other of two predefined phage coat proteins. This approach allow the selection of identification of protein sequence that can be differentially expressed or displayed on surface or recombinant phage, and consequently selected from a phage display library with different efficiency.

In the present case, a phage library was constructed in a pDD phagemid by cloning the variable regions of human heavy and light chain immunoglobulins. The library was panned against VZV protein extracts and selected clones were subsequently tested in cell-based assays for determining the ones that present VZV neutralizing activities, as shown in the Examples.

The DNA sequence that encode the two most promising clones, named DDF-VZV1 and DDF-VZV2, were determined and then cloned in an appropriate vector for bacterial expression. The VZV-neutralizing activity of these Fabs have been tested as both fusion proteins on the surface of recombinant phage purified from bacterial cell cultures or as affinity-purified, recombinant human Fabs, using in vitro models for VZV infection.

The present invention provides novel protein sequences that are capable of binding and neutralizing VZV and that include specific CDRs (Complementarity Determining Regions) identified in the Fabs DDF-VZV1 or DDF-VZV2. In particular, each of the HCDR3s (CDR3 of the heavy chain variable region) of the invention (SEQ ID NO.: 5 and SEQ ID NO.: 12) characterizes the antigen-binding portion of DDF-VZV1 and DDF-VZV2, respectively.

The HCDR3 of an antibody can be considered as characterizing the antigen-binding portion of such antibody that is capable of binding an antigen and, consequently, exerting a biological activity (e.g. binding and neutralizing VZV, as shown in the Examples). Even though, several or all CDRs of an antibody are generally required for obtaining a complete antigen-binding surface, HCDR3 is the CDR showing the highest differences between antibodies not only with respect to sequence but also with respect to length. In fact, the diversity of HCDR3 sequence and length is fundamental for determining the specificity for most antibodies (Xu J and Davies M, 2000; Barrios Y et al. 2004; Bond C et al., 2003).

Proteins containing a specific HCDR3 of the Invention as VZV binding moiety, in combination or not with other CDRs from the same Fab in which such HCDR3 was identified, can be generated within an antibody protein framework (Knappik A et al., 2000). Combinations of CDRs can be linked to each other in very short proteins that retain the original binding properties, even within a protein framework unrelated to antibody structure and without disrupting the original binding activity (Ladner R, 2007; Kiss C et al., 2006).

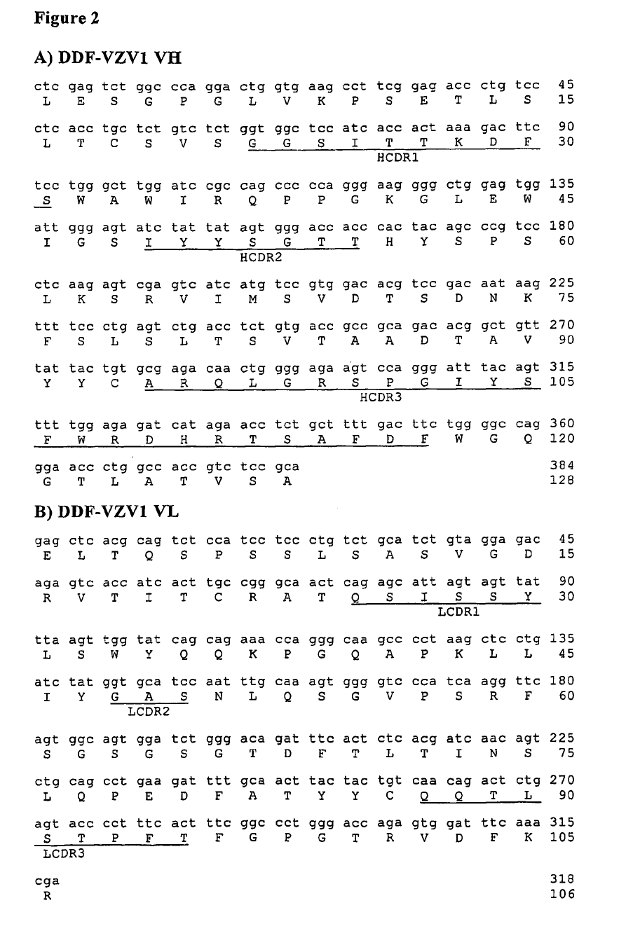

In one embodiment, the present invention provides a protein comprising a sequence having at least 90% identity with the HCDR3 of the Fab DDF-VZV1. Together with the HCDR1 and HCDR2, (SEQ ID NO.: 3 and SEQ ID NO.: 4; FIG. 2A), this HCDR3 is included in the variable region of the heavy chain of DDF-VZV1 Fab (DDF-VZV1 VH; SEQ ID NO.: 2). The variable region of a light chain that forms this Fab (DDF-VZV1 VL: SEQ ID NO.: 7), as well its specific LCDRs, (CDRs of the light chain variable region), have been determined (FIG. 2B).

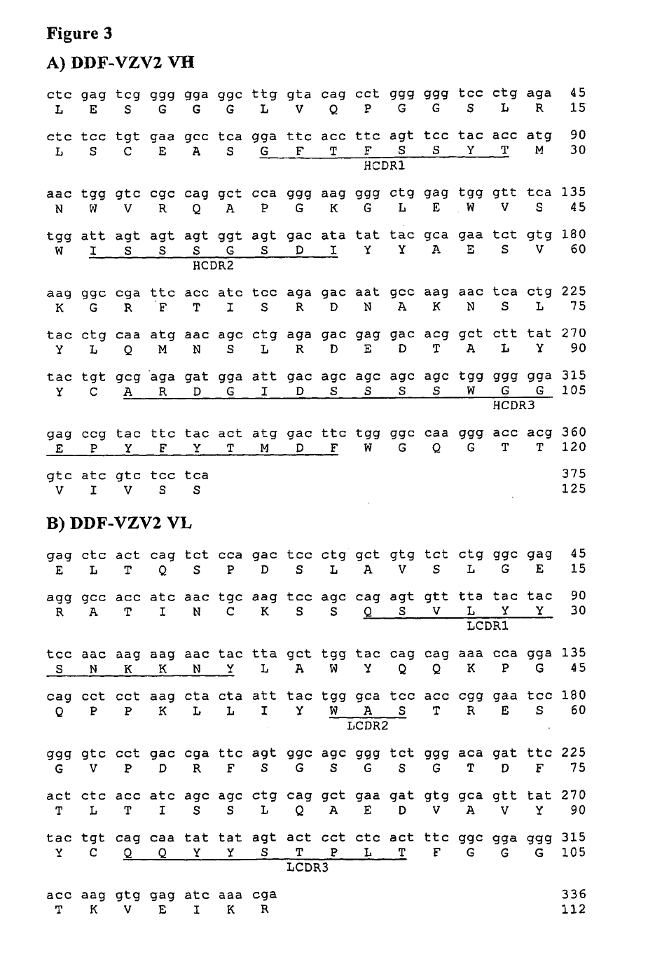

In another embodiment, the present invention provides protein comprising a sequence having at least 90% of identity with the HCDR3 of the Fab DDF-VZV2. Together with the HCDR1 and HCDR2 (SEQ ID NO.: 10 and SEQ ID NO.: 11; FIG. 3A), this HCDR3 is included in the variable region of the heavy chain of DDF-VZV2 (DDF-VZV2 VH; SEQ ID NO.: 9). The variable region of a light chain that forms this Fab (DDF-VZV1 VL; SEQ ID NO.: 14), as well its specific LCDRs, have been determined (FIG. 3B).

If the proteins of the Invention are based on the sequence of DDF-VZV1, they should comprise a sequence having at least 90% identity to SEQ ID NO.: 5. In particular, they should comprise a sequence having at least 90% identity with SEQ ID NO.: 2. More in particular, such proteins should also include one or more sequences selected from the group consisting of SEQ ID NO.:3 and SEQ ID NO.: 4.

Alternatively, if the proteins of the Invention are based on the sequence of DDF-VZV2, they should comprise a sequence having at least 90% identity to SEQ ID NO.: 12. In particular, they should comprise a sequence having at least 90% identity with SEQ ID NO.: 9. More in particular, such proteins should also include one or more sequences selected from the group consisting of SEQ ID NO.: 10 and SEQ ID NO.: 11.

Further embodiments of the Invention are the DNA sequences encoding the variable region of heavy and light chains of both Fabs, in particular those having at least 90% of identity with the original DNA sequences that have been cloned and determined for the variable regions of DDF-VZV1 (SEQ ID NO.: 1 for the heavy chain; SEQ ID NO.: 6 for the light chain) and DDF-VZV2 (SEQ ID NO.: 8 for the heavy chain; SEQ ID NO.:13 for the light chain). These DNA sequences (or selected portions, such as those encoding the isolated HCDRs and LCDRs which can be easily determined from FIGS. 2 and 3) can be transferred in other vectors for expressing them within one of the known formats for recombinant antibodies (e.g. full, affinity-matured, CDR-grafted, or fragments) or fusion proteins to which they confer VZV binding and neutralizing properties.

Wherever a level of identity is indicated, this level of identity should be determined on the full length of the relevant sequence of the invention.

The variable region of the heavy and light chains forming either DDF-VZV1 or DDF-VZV2 (or selected portions, such as the isolated HCDRs and LCDRs) can be comprised within an antibody having a specific isotype, in particular within a fully human recombinant antibody. This antibody may comprise the VL and VH sequences of either DDF-VZV1 or DDF-VZV2 as light and heavy chains variable regions in the natural conformation of a tetrameric complex formed by two light and two heavy chains. When a fully human antibody is desirable, the antibody should further comprise a heavy chain constant region selected from the group consisting of human IgG1, IgG2, IgG3, IgG4, IgM, IgA and IgE constant regions. The IgG isotype, for example, is the antibody format of almost all approved therapeutic antibodies (Laffly E and Sodoyer R, 2005). However, antigen binding portions isolated from a human IgG1 can be transferred on a human IgA sequence and the resulting recombinant antibody maintained the activity of the original IgG1, as recently shown with an antibody capable of inhibiting HIV infection (Mantis N et al., 2007).

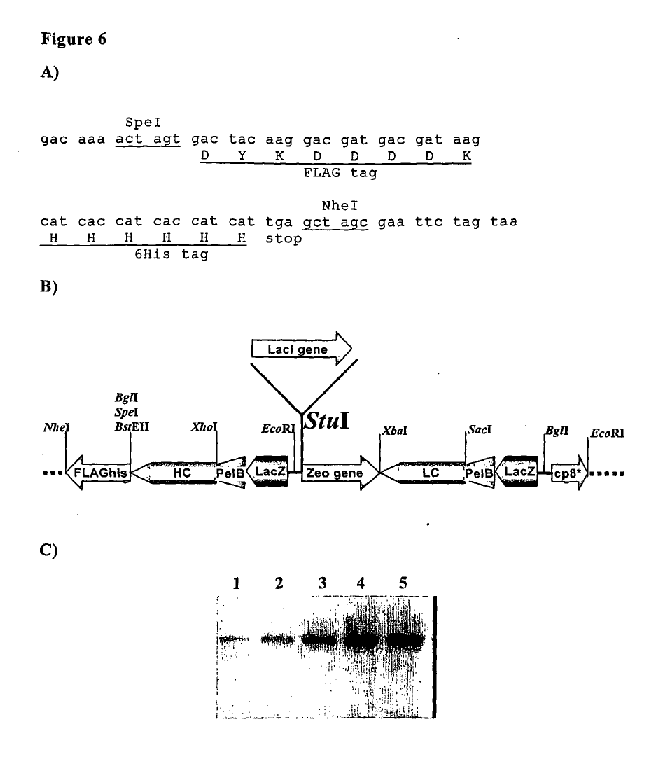

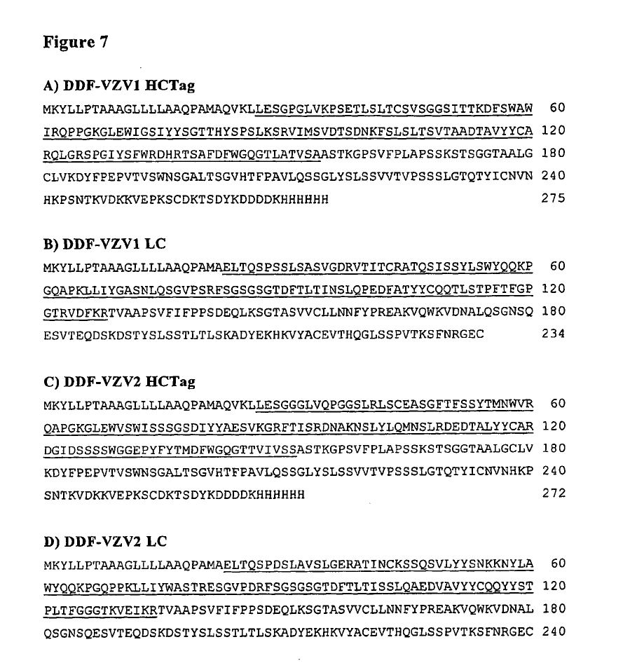

Alternatively, the variable region of the heavy and light chains forming either DDF-VZV1 or DDF-VZV2 (or selected portions, such as the isolated HCDRs and LCDRs) can be comprised in any other protein format for functional antibody fragments, as described in the literature under different names such as Scfv (single-chain fragment variable), Fab (variable heavy/light chain heterodimer), diabody, peptabody, VHH (variable domain of heavy chain antibody), isolated heavy or light chains, bispecific antibodies, and other engineered antibody variants for non-/clinical applications (Jain M et al., 2007; Laffly E and Sodoyer R, 2005). Recombinant variants of DDF-VZV1 or DDF-VZV2 were produced using a pDD-compatible expression vector called pDLac-FLAGhis in the form of tagged Fabs (FIG. 6B for a schematic map of the relevant DNA fragment; SEQ ID NO.: 18 for an example of DNA sequence for such fragment that has been generated for producing DDF-VZV1). The vectors based on pDLac-FLAGhis that include DDF-VZV1 and DDF-VZV2 tagged versions were generated and used for producing DDF-VZV1 HCtag together with DDF-VZV1 LC (SEQ ID NO.: 19-22; FIG. 7A and B), or DDF-VZV2 HCtag together with DDF-VZV2 LC (SEQ ID NO.: 23-26; FIGS. 7C and D).

Additional antibodies and antibody fragments can be generated using the sequences of either DDF-VZV1 or DDF-VZV2 through a process for shuffling light chains. In fact, several different antibodies can be generated and tested for specific biological activity using a single heavy chain variable domain VH (such as the one of either DDF-VZV1 or DDF-VZV2) which is combined with a library of VL sequences, for example using common phage display technologies or those described in WO 07/007,154. This approach may allow determining VH/VL combinations with improved properties in terms of affinity, stability, specificity, and/or recombinant production (Ohlin M et al., 1996; Rojas G et al., 2004; Suzuki K et al., 2007).

Moreover, it is known that antibodies may be modified in specific positions in order to have antibodies with improved features, in particular for clinical applications (such as better pharmacokinetic profile or higher affinity for an antigen). These changes can be made in the CDRs and/or framework of either DDF-VZV1 or DDF-VZV2. The sequence can be determined by applying any of the dedicated technologies for the rational design of antibodies that make use of affinity maturation and other methods (Kim S et al., 2005; Jain M et al., 2007).

Antibody-based strategies for developing new bioactive peptides also showed the feasibility of synthetizing CDR-derived peptides that contain L-amino acids and/or D-amino acids. These molecules can maintain the specific activity of the original activity with a more appropriate pharmacological profile (Levi M et al., 2000; Wijkhuisen A et al., 2003). Thus, each of the HCDR3 of the Invention, as well as sequences highly similar to them, fusion proteins containing them, and synthetic peptides derived from them (e.g. containing D-amino acids or in the retro-inverso conformation), can be tested and used as VZV-binding proteins.

The protein of the invention may be provided as antibodies, antibody fragments, bioactive peptides, or fusion proteins that binds and neutralize VZV. These alternative proteins should maintain, if not enhance such properties as determined for DDF-VZV1 and DDF-VZV2 Fabs. In the case of fusion proteins, the heterologous protein sequences can be located in the N- or C-terminal position to the VZV-specific moiety (e.g. the specific HCDR3 or variable region of an antibody fragment), without affecting its correct expression and biological activity.

The term “heterologous protein” indicates that a protein sequence is not naturally present in the N- or C-terminal position to the VZV-specific moiety (e.g. an antibody fragment). The DNA sequence encoding this protein sequence is generally fused by recombinant DNA technologies and comprises a sequence encoding at least 5 amino acids. This heterologous sequences present is generally chosen for providing additional properties to the VZV-specific fusion protein. Examples of such additional properties include better means for detection or purification, additional binding moieties or biological ligands, or post-translational modification of the fusion protein (e.g. phosphorylation, glycosylation, ubiquitination, SUMOylation, or endoproteolytic cleavage).

Alternatively (or additionally to the fusion to one or more heterologous protein sequences), the activity of a protein of the invention may be improved with the conjugation to different compound such as therapeutic, stabilizing, or diagnostic agents. Examples of these agents are detectable labels (e.g. a radioisotope, a fluorescent compound, a toxin, a metal atom, a colloidal metal, a chemiluminescent compound, a bioluminescent compound, or an enzyme) that can be bound using chemical linkers or polymers. The VZV-specific biological activity of a protein of the invention may be improved by the fusion with a compound, such as a polymer altering the metabolism and/or the stability in diagnostic or therapeutic applications. The therapeutic activity may be improved by the fusion with another therapeutic protein, such as another antiviral protein or a protein altering cell metabolism and/or activity.

Means for choosing and designing protein moieties, ligands, and appropriate linkers, as well as methods and strategies for the construction, purification, detection and use of fusion proteins are provided in the literature (Nilsson et al., 1997; “Applications Of Chimeric Genes And Hybrid Proteins” Methods Enzymol. Vol. 326-328, Academic Press, 2000; WO01/77137) and are commonly available in clinical and research laboratories. For example, the fusion protein may contain sequences recognized by commercial antibodies (including tags such as polyhistidine, FLAG, c-Myc, or HA tags) that can facilitate the in vivo and/or in vitro identification of the fusion protein, or its purification. Other protein sequences can be easily identified by direct fluorescence analysis (as in the case of Green Fluorescent Protein), or by specific substrates or enzymes (using proteolytic sites, for example).

The stability of the VZV-specific antibodies, antibody fragments, and fusion proteins may be improved with the fusion with well-known carrier proteins, such as phage coat protein (cp3 or cp8, isolated or included in a recombinant phage), Maltose Binding Protein (MBP), Bovine Serum Albumin (BSA), or Glutathione-S-Transferase (GST).

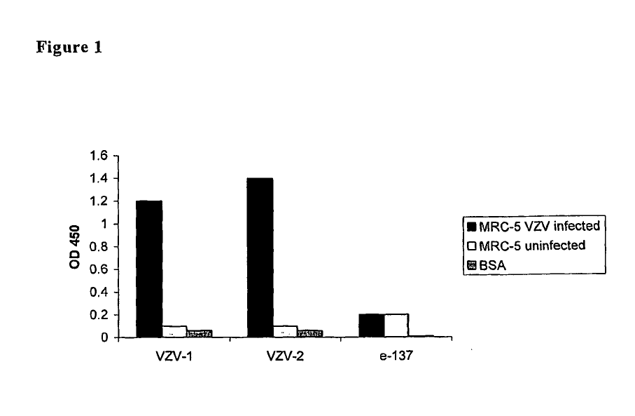

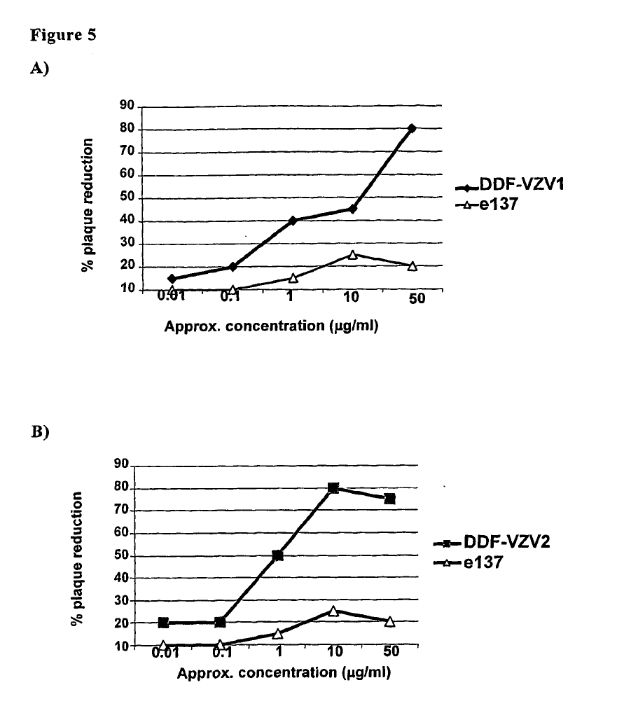

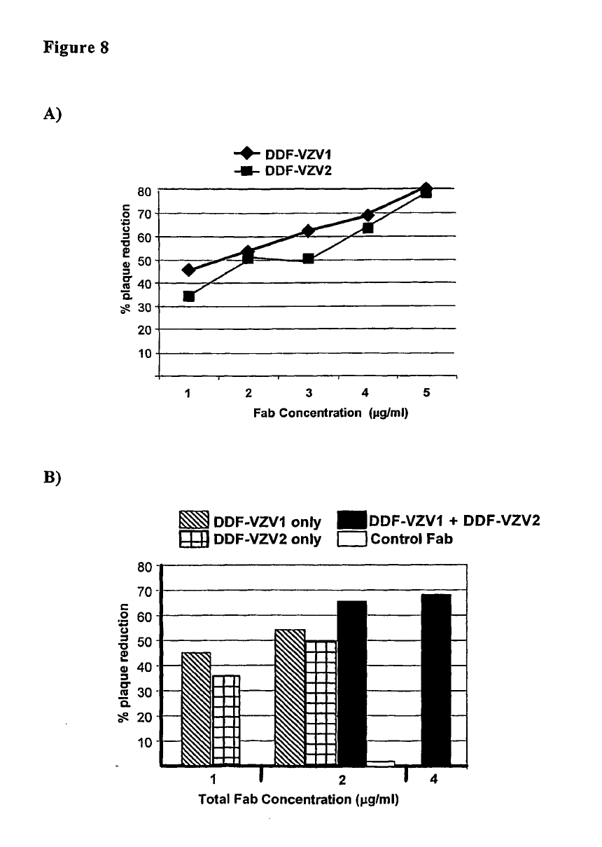

The proteins of the Invention can be also used for characterizing neutralizing antigens on VZV envelope. In fact, DDF-VZV1 and DDF-VZV2 have been initially cloned because of their specific binding to cell extracts derived from VZV-infected cell lines in ELISA (FIG. 1) and their capability to neutralize VZV infection was also determined by an in vitro neutralization assay using a VZV reference strain (FIGS. 5 and 8). Consequently, the protein of the invention can be used for defining other VZV-binding proteins (in form of full antibodies, Fabs and other antibody fragments, bioactive peptides, or fusion proteins, for example) that compete with DDF-VZV1 or DDF-VZV2. Such competing proteins may simply contain any of the HCDR3 sequences defined above, optionally together with HCDRs and LCDRs in part or completely different from those identified in the original DDF-VZV1 or DDF-VZV2 sequences.

Such competing proteins (as for the antibodies, antibody fragments, bioactive peptides, or fusion proteins of the Invention) can be screened and isolated by demonstrating their capability to compete with the protein of the invention, and then their capability of neutralizing VZV infection, as determined by any relevant assay, as described in the Examples or the literature. The Background of the Invention provides several references on the different approach for determining similar activities. In particular, antibodies, antibodies fragments, and the other proteins of the Invention can be tested in assays used for characterizing the VZV-specific biological activity and epitope for antibody fragments (Kausmally L et al., 2004; Drew P et al., 2001; Suzuki K et al., 2007), murine or human monoclonal antibodies (Grose C et al., 1983; Montalvo E and Grose C, 1986; Sugano T et al., 1987; Forghani B et al., 1994; Lloyd-Evans P and Gilmour J, 2000), and non-/human sera (Fowler W et al., 1995; Haumont M et al., 1997; Garcia-Valcarcel M et al., 1997).

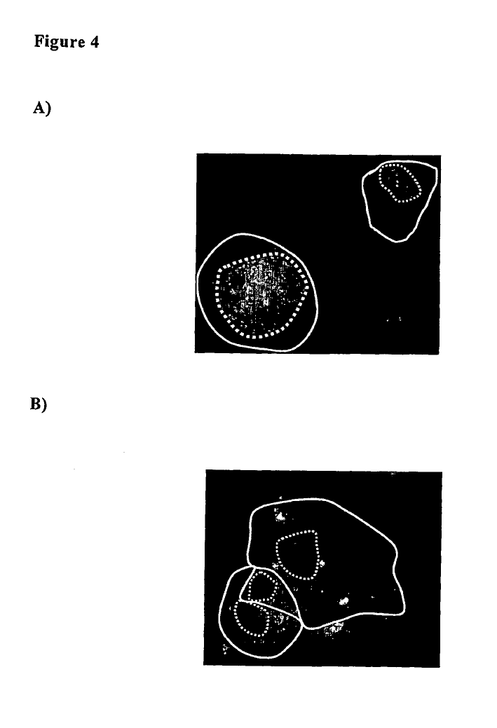

The immunofluorescence studies (FIG. 4) showed that DDF-VZV1 and DDF-VZV2 recognize distinct VZV antigens, as done in the past for other antibodies and antibody fragments (Suzuki K et al., 2007; Wu L and Forghani B, 1997; Grose C et al., 1983). The literature provides several examples of technologies using which the VZV antigen and the specific epitope that is recognized by each Fab can be determined and compared to those determined in the past. For example, ELISA, immunoprecipitation, or Western Blot using VZV proteins, and related truncated variants or synthetic peptides, have used to determine relevant epitopes (Krah D, 1996, Sauerbrei A and Wutzler P, 2006). In particular such epitopes have been identified within Glycoprotein E or Glycoprotein B (Fowler W et al., 1995; Haumont M et al., 1997; Garcia-Valcarcel M et al., 1997; Kjartansdottir A et al., 1996), and Glycoprotein L:Glycoprotein H complex (Forghani B et al., 1994; Yokoyama T et al., 2001; Suzuki K et al., 2007).

More extensive characterization and validation for VZV-related prophylactic, diagnostic, and therapeutic uses of the protein of the Invention can then be performed using one or more of the in vitro or in vivo assays (tissue- or cell-based assays, disease models established in rodents) that are disclosed in the literature for studying VZV pathogenesis and immunobiology, as summarized in the Background of the Invention (Forghani B et al., 1994; Vafai A et al., 1991; Wu and Forghani, 1997; Fowler W et al., 1995; Maple P et al., 2006; Matsuo K et al., 2003; Kutinova L et al., 2001; Massaer M et al., 1999; Haumont M et al., 1997; Grose C, 2006; Baiker 2004; Ku C et al., 2005; Taylor S and Moffat J., 2005; Zerboni L et al., 2005).

Further objects of the inventions are the nucleic acids encoding any of the antibodies, antibody fragments, fusion proteins or isolated CDRs defined above. The examples provide such sequences in particular as encoding the full variable regions of DDF-VZV 1 or DDF2-VZV2 heavy and light chains (SEQ ID NO.: 1, 6, 8, and 13). The nucleic acid should have at least 90% identity with SEQ ID NO.:1, SEQ ID NO.:6, SEQ ID NO.:8 and/or SEQ ID NO.:13. Such sequences, in particular those within them that are associated to specific CDRs (see FIGS. 2 and 3), can be comprised in vector and DNA expression cassette, for example being operably linked to a promoter in an expression vector or cloned in a pDD-based phagemid, as well as in any other phagemid. Thus, the recombinant phage comprising a phagemid vector that expresses a protein of the Invention (as shown in the Examples) can be used as means for detecting and/or neutralizing VZV infection.

When a fully human antibody is desirable, the expression vector should further comprise a heavy chain constant region selected from the group consisting of IgG1, IgG2, IgG3, IgG4, IgM, IgA and IgE constant regions. The nucleic acid sequences encoding the relevant variable regions of the heavy and light chain of interest should be appropriately cloned in the expression cassette of a vector or of distinct vectors where they are operably linked to appropriate regulatory sequences (e.g. promoters, transcription terminators). The expression cassette should include a promoter, a ribosome binding site (if needed), the start/stop codons, and the leader/secretion sequence, that can drive the expression of a mono- or bicistronic transcript for the desired protein.

The antibody, the antibody fragments, the HCDRs, the fusion proteins, and any other compound of the invention defined above as being capable of binding and neutralizing VZV can be produced using such vectors for transforming the appropriate host cells and the well-established technologies that allow expressing them as recombinant proteins. These preparations should provide a sufficient amount of recombinant protein (from the microgram to the milligram range) to perform a more extensive characterization and validation for VZV-related prophylactic, diagnostic, and therapeutic uses.

The pDD-based phagemids in which the sequences of the invention have been cloned and characterized by means of the corresponding recombinant phage, contain DNA sequences that can be transferred (in part or totally) into vectors where the original Fabs, or protein sequences derived from them, can be appropriately expressed as recombinant proteins in host cells (as shown in the Examples with the pDLac-FLAGhis system). The vectors should allow the expression of the recombinant protein in the prokaryotic or eukaryotic host cells under the control of transcriptional initiation/termination regulatory sequences, which are chosen to be constitutively active or inducible.

The host cells comprising the nucleic acids of the invention can be prokaryotic or eukaryotic host cells and should allow the secretion of the desired recombinant protein. A cell line substantially enriched in such cells can be then isolated to provide a stable cell line. Methods for producing such proteins include culturing host cells transformed with the expression vectors comprising their coding sequences under conditions suitable for protein expression and recovering the protein from the host cell culture.

These nucleic acids, recombinant phage, and host cells can be used for producing a protein of the Invention by applying common recombinant DNA technologies. Briefly, the desired DNA sequences can be either extracted by digesting the phagemid with restriction enzymes, or amplified using the original phagemid as a template for a Polymerase Chain Reaction (PCR) and the PCR primers for specifically amplifying full variable regions of the heavy and light chains or only portions of them (such as an HCDR3).

Such DNA fragments can be then transferred into more appropriate vectors for further modification and/or expression into prokaryotic or eukaryotic host cells, as described in many books and reviews on how to clone and produce recombinant proteins, including some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

For eukaryotic hosts (e.g. yeasts, insect or mammalian cells), different transcriptional and translational regulatory sequences may be employed, depending on the nature of the host. They may be derived from viral sources, such as adenovirus, bovine Papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for the transient (or constitutive) repression and activation and, consequently, for modulating gene expression. During further cloning steps, the sequence encoding the antibody or the fusion protein can be adapted and recloned in other vectors for specific modifications at the DNA level only at both the DNA and protein level that can be determined, for example, using software for selecting the DNA sequence in which the codon usage and the restriction sites are the most appropriate for cloning and expressing a recombinant protein using specific vectors and host cells (Rodi D et al., 2002; Grote A et al., 2005; Carton J et al., 2007). Protein sequences can also be added in connection to the desired antibody format (Scfv, Fab, fully human antibody, etc.), or to the insertion, substitution, or elimination of one or more internal amino acids.

These technologies can also be used for further structural and functional characterization and optimization of the therapeutic properties of antibodies (Kim S et al., 2005), or for generating vectors allowing their stable in vivo delivery (Fang J et al., 2005). For example, recombinant antibodies can also be modified at the level of structure and/or activity by choosing a specific Fc region to be fused to the variable regions (Furebring C et al., 2002; Logtenberg T, 2007), by generating recombinant single chain antibody fragments (Gilliland L et al., 1996), by fusing stabilizing peptide sequences (WO 01/49713), or by adding radiochemicals or polymers to chemically modified residues (Chapman A et al., 1999).

The DNA sequence coding for the displayed and selected protein sequence, once inserted into a suitable episomal or non-homologously or homologously integrating vector, can be introduced in the appropriate host cells by any suitable means (transformation, transfection, conjugation, protoplast fusion, electroporation, calcium phosphate precipitation, direct microinjection, etc.) to transform them. Important factors to be considered when selecting a particular plasmid or viral vector include: the ease with which host cells that contain the vector may be recognized and selected from those cells which do not contain the vector; the number of copies of the vector which are desired in a particular host; and whether it is desirable to “shuttle” the vector between host cells of different species.

The cells which have been stably transformed by the introduced DNA can be selected by also introducing one or more markers which allow for selection of host cells which contain the expression vector. The marker may also provide for phototrophy to an auxotropic host, biocide resistance, e.g. antibiotics, or heavy metals such as copper, or the like, and it may be cleavable or repressed if needed. The selectable marker gene can either be directly linked to the DNA gene sequences to be expressed, or introduced into the same cell by co-transfection. Additional transcriptional regulatory elements may also be needed for optimal expression.

Host cells may be either prokaryotic or eukaryotic. Amongst prokaryotic host cells, the preferred ones are B. subtilis and E. coli. Amongst eukaryotic host cells, the preferred ones are yeast, insect cells (using baculovirus-based expression systems), or mammalian cells, such as human, monkey, mouse, insect (using baculovirus-based expression systems) and Chinese Hamster Ovary (CHO) cells, because they provide post-translational modifications to protein molecules, including correct folding or certain forms of glycosylation at correct sites. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast recognize leader sequences in cloned mammalian gene products and secrete peptides bearing leader sequences (i.e., pre-peptides).

Mammalian cell lines available as hosts for expression are known in the art and include many immortalized cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Per.C6, Bowes melanoma and human hepatocellular carcinoma (for example Hep G2) cells and a number of other cell lines. In the baculovirus system, the materials for baculovirus/insect cell expression systems are commercially available in kit form (e.g. commercialized by Invitrogen).

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may proliferate using tissue culture techniques appropriate to the cell type. A cell line substantially enriched in such cells can be then isolated to provide a stable cell line. The host cells can be further selected on the basis of the expression level of the recombinant protein.

In the case of immunoglobulin variable chains (in particular human immunoglobulin variable chains) that are isolated using phage display technologies, an important modification is the conversion of the selected Fab or scFV into a full immunoglobulin protein having a preferred isotype and constant region. This kind of modification allows, for example, generating full human monoclonal antibodies of all isotypes constructed from phage display library-derived single-chain Fv or Fabs and expressing in mammalian or insect cells. As widely described in the literature (Persic L et al., 1997; Guttieri M et al., 2003), the vectors are specifically designed for expressing antibodies, allowing the fusion of this sequence to constant (Fc) regions of the desired isotype (for example, human IgG gammal). The antibodies or fusion proteins can be expressed as recombinant proteins in prokaryotic organisms (e.g. Escherichia coli; Sørensen H and Mortensen K, 2005; Venturi M et al., 2002), plants (Ma J et al., 2005), or eukaryotic cells, that allow a high level of expression as transient or stable transformed cells (Dinnis D and James D, 2005). This would be required in particular when the characterization of the antibodies has to be performed using more demanding and/or in vivo assays.

The literature provides different strategies for expressing a protein as a Fab or a similar format for antibody fragments, in prokaryotic host cells, as reviewed in articles and chapters of books (“Phage display: A Practical Approach”, vol. 266, ed. Clackson and Lowman H, Oxford Univ. Press, 2004; “Phage Display: A Laboratory Manual”, ed. Burton D et al., CSHL Press, 2001; Corisdeo S and Wang B, 2004; Benhar 1, 2001).

When the protein, especially an antibody, is expressed in eukayotic host cells (mammalian cell lines, in particular), different vector and expression systems have been designed for generating stable pools of transfected cell lines (Aldrich T et al., 2003; Bianchi A and McGrew J, 2003). High level, optimized, stable expression of recombinant antibodies has been achieved (Schlatter S et al., 2005), also due to optimization of cell culture conditions (Grunberg J et al., 2003; Yoon S et al., 2004) and by selecting or engineering clones with higher levels of antibody production and secretion (Bohm E et al., 2004; Butler M, 2005).

The antibody, the antibody fragments, the bioactive peptide, the fusion protein, and any other protein defined above as being capable of binding and neutralizing VZV can be purified using the well-established technologies that allow the isolation of either non-/recombinant proteins from cell culture or from synthetic preparations, i.e. any conventional procedure involving extraction, precipitation, chromatography, electrophoresis, or the like. Methods for antibody purification can make use of immobilized gel matrix contained within a column (Nisnevitch M and Firer M, 2001; Huse K et al., 2002; Horenstein A et al., 2003) and in particular on the general affinity of antibodies for substrates such protein A, protein 0, or synthetic substrates (Verdoliva A et al., 2002; Roque A et al., 2004), as well as by antigen- or epitope-based affinity chromatography (Murray A et al., 2002; Jensen L et al., 2004). After washing, the protein is eluted from the gel by a change in pH or ionic strength. Alternatively, HPLC (High Performance Liquid Chromatography) can be used. The elution can be carried out using a water-acetonitrile-based solvent commonly employed for protein purification.

The antibody, the antibody fragments, the bioactive peptides, the fusion proteins, and any other compound defined above as being capable of binding and neutralizing VZV can be used for detecting, treating, inhibiting, preventing, and/or ameliorating VZV infection. To this purpose, such compounds can be used for preparing diagnostic, therapeutic, or prophylactic compositions for the medical management of VZV infection and VZV-related diseases.

These compositions may comprise an antibody, antibody fragment, fusion proteins, CDRs, and any other compound defined above on the basis of the sequence and activity of human DDF-VZV1 and DDF-VZV2. The compositions may further comprise a different VZV-neutralizing antibody or antibody fragment, an intravenous immunoglobulins (IVIg) preparation, a steroid, and/or an antiviral compound. The different VZV-neutralizing antibody or antibody fragment should be characterized by a different epitope, such as the ones already described in the literature. In fact, the literature shows many examples in which, when two or more antibodies directed to a viral or human target are combined in a pharmaceutical composition, the resulting composition may have an improved therapeutic efficacy due not to a simple additive effect but to a specific synergic effect (Logtenberg T, 2007).

Pharmaceutical compositions may optionally comprise any pharmaceutically acceptable vehicle or carrier. These compositions may further comprise (or may be administered together with) any additional therapeutic or prophylactic agent, such as vaccines, immunomodulating intravenous immunoglobulin preparations, steroids, or antiviral compounds. The literature provides some examples of such compounds acting on VZV replication and already tested in humans, alone or in combination with intravenous immunoglobulin preparations (Huang Y et al., 2001; Carby M et al., 2007; Koren G et al., 2002). Moreover, recent literature suggests that human monoclonal antibodies can be used for supplementing (and replacing, if possible) present treatments such as intravenous immunoglobulin preparations, steroid, and/or antiviral compounds, giving the opportunity to reduce frequency and/or dosage of such pharmaceutical compositions (Bayry J et al., 2007).

The compositions that comprise any of the proteins (e.g. antibodies, antibody fragments, fusion proteins, bioactive peptides) and of the nucleic acids defined above can be administered to an individual with a VZV-related diagnostic, therapeutic, or prophylactic purpose. These compositions can be administered as means for VZV-specific passive immunization which provide therapeutic compounds (in particular therapeutic antibodies or therapeutic antibodies fragments) that, by targeting VZV virions, can inhibit the propagation of the virus in the treated patient, and potentially block the outbreak of a viral infection in the population.

Depending on the specific use, the composition should provide the compound to the human subject (being an infant, a pregnant woman, an elderly individual, or any other individual that is infected by VZV or considered at risk for VZV due to an hospitalization, an immunosuppressive or chemotherapeutic treatment, or contact with a VZV-infected individual) for a longer or shorter period of time. To this purpose, the composition can be administered, in single or multiple dosages and/or using appropriate devices, through different routes: intramuscularly, intravenously, subcutaneously, topically, mucosally, by a nebulizer, an inhaler, or as eyedrops, in non-/biodegradable matrix materials, or using particulate drug delivery systems such as microbeads.

In particular, the composition may allow topical and ocular administration, that represent a useful approach given the presence of VZV in skin and eye (Arvin A, 1996; Quinlivan M and Breuer J, 2006; Liesegang T, 2004). Moreover, antibodies and antibody fragments can be effective when applied topically to cornea (Brereton H et al., 2005; Nwanegbo E et al., 2007).

A pharmaceutical composition should provide a therapeutically or prophylactically effective amount of the compound to the subject that allows the compound to exert its activity for a sufficient period of time, in particular for topical administration, given the presence of the virus in cutaneous rash associated to secondary viremia. The desired effect is to improve the status of the patient by controlling VZV infection, reactivation, and/or re-infection, and by reducing at least some of the clinical manifestations of VZV infection. For example, the composition should be administered at an effective amount from about 0.005 to about 50 mg/kg/body weight, depending on the route of administration the number of administered doses, and the status of the individual.

In the case of composition having diagnostic uses, the compound should be detected using technologies commonly established in the clinical and research laboratories for detecting virus in biological samples (e.g. ELISA or other serological assays), or, when administered to a subject in vivo, at least 1, 2, 5, 10, 24, or more hours after administration. The detection of VZV can be performed, using the proteins of the invention, in substitution or coupled to the known means and procedures that have been established for monitoring chronic or acute VZV infection in at risk populations of both immunocompetent and immunocompromised hosts.

The proteins of the invention can be also used for the preparation of a composition for detecting, treating, inhibiting, preventing, and/or ameliorating VZV infection, as well as VZV-related diseases. These diseases may result from the complications of VZV infection (Dworkin R et al., 2007; Weinberg J, 2007).

As indicated in the Background, there is a large number of Herpes zoster complications of Herpes Zoster having neurological, ocular, or visceral effects that can have dramatic and debilitating effects, as in the case of post-herpetic neuralgia (Oxman M et al., 2006; Liesegang T, 2004). Moreover, re-activation of VZV and related complications has also been found in cancer patients (Sandherr M et al., 2006) or patients affected by inflammatory connective tissue diseases, and in general in patients under immunosuppressive treatments such as corticosteroids, or chemotherapy and other antibody-based immunosuppressive regimens.

A method for treatment, prophylaxis, or diagnosis of VZV, or of VZV-related disease can comprise the administration of a protein or of a nucleic acid as above defined. The method may further comprise the administration of a different VZV-neutralizing antibody or antibody fragment, an intravenous immunoglobulins (IVIg) preparation, a steroid, and/or an antiviral compound.

The clinical development and use should be based on the characterization of the antibody pharmacokinetics and pharmacodynamics (Lobo E et al., 2004), the preclinical and clinical safety data (Tabrizi M and Riskos L, 2007), and compliancy to official requirements for commercial manufacturing scale formulation and analytical characterization of therapeutic recombinant antibodies (Harris R et al., 2004).

The invention will now be described by means of the following Examples, which should not be construed as in any way limiting the present invention.

### EXAMPLES

### Example 1

### Expression and Selection of Human Fabs Binding VZV

### Protein Extracts in ELISA

Materials & Methods

Library Construction

The cDNA encoding for heavy and light chains of human IgG1 was obtained from lymphocytes obtained from a VZV-seropositive individual according to the literature (Burioni et al., 1998; “Phage Display: A laboratory Manual”, Burton D R et al., CSHL Press, 2001). The phage library was constructed using a cloning cassette compatible with a pDD vector according to the technology described in the PCT patent application WO07/007,154, and the Fabs were expressed on the surface of the recombinant phage in the library.

The selection of human Fabs through the panning of the pDD-based Fab library and the sequencing of the positive clones was performed as described in the literature (Burioni R et al. 1998).

The CDRs of the specific Fabs were defined by comparing the predictions and sequence alignments provided by IMGT/V-QUEST (Giudicelli V et al., 2004) and other databases containing protein sequences of human antibodies, such as those provided by the European Bioinformatics Institute and searchable using FASTA (http://www.ebi.ac.uk/fasta33/index.html).

Preparation of Protein Extracts from Cultured Human Cells

The cell line MRC-5 (ATCC Acc. No. CCL-171), which is a human embryonic lung fibroblast cell line commonly used for VZV isolation and propagation, was used for the preparation of the VZV-specific material for panning the phage display library and testing the Fabs in ELISA. MRC-5 cells are maintained in Modified Eagle Medium containing 10% of foetal bovine serum inactivated (FBS), 50 μg/ml of penicillin, 100 μg/ml streptomycine and 2 mM L-glutamine.

The cells (VZV-infected or uninfected MRC-5) were scraped and resuspended in 250 ml of lysis buffer (50 mM Tris-HCl pH 8.0; 150 mM NaCl; 0.02% Sodium Azide; 0.5% Triton-X), incubated for 20 minutes on ice, then centrifuged 12000 rpm for 2 minutes at 4° C. The protein concentration of the resulting supernatants was determined in duplicates using BCATM Protein Assay Kit (Pierce). Protein concentration of unknown samples was determined and reported with reference to a serial dilution of Bovine Serum Albumin (BSA) at known concentrations (0 mg/ml=Blank−2.000 mg/ml=max. value). The absorbance of all the samples was measured with the spectrophotometer set to 540 nm.

Panning and ELISA Using Protein Extracts

The protein coating was performed on 96-well plates using the following antigens: cell lysate of MRC-5 human fibroblasts infected with VZV (ELLEN strain; ATCC; Acc. No. VR-586); a commercial preparation of Influenza virus antigens (Virion Ltd.); cell lysate of uninfected MRC-5; Bovine Serum Albumin (BSA). Each sample was diluted in carbonate buffer (100 nanograms of total protein in 25 □l final volume per well) and the plate was incubated overnight at 4° C. After washing with distilled water, the plate was blocked by incubation in PBS with 1% BSA for 1 hour at 37° C.

The ELISA was performed using 40 □l of undiluted sample containing the following Fabs using a protocol disclosed in the literature: DDF-VZV1, DDF-VZV2, and e137, an unrelated Fab prepared (Bugli F et al., 2001). The Fabs were tested in duplicate wells. After incubation with each Fab for 1 hour at 37° C. and five washings with PBS with 0.1% Tween-20, 40 μl goat anti-human Fab, peroxidase-conjugate (Sigma; Cat no. A0293) were added and incubated for 1 hour at 37° C. Plate washing was repeated as above and enzymatic reaction was developed by adding 40 of substrate (TMB Substrate Kit; Pierce) to each well. ELISA reactions were developed for 15 minutes at 37° C. Enzyme activity was stopped by adding stop solution (H₂SO₄) and the absorbance measured with a spectrophotometer set to 450 nanometers.

Fab Preparation for ELISA and Neutralization Assay

The protocol was similar to those described in the literature using pDD or other phagemids such as pGem variants (“Molecular Cloning: A Laboratory Manual”, Sambrook et al., Cold Spring Harbor Press, NY, 1989; Burioni R et al., 1998; Burioni R et al., 2001; WO07/007,154).

Briefly, individual E. coli clones of the library were grown in 200 ml Super Broth (SB; 3.5% bacto-tryptone, 2% yeast extract, 0.5% NaCl) medium with antibiotics and supplemented with IPTG, harvested and washed with phosphate-buffered saline (PBS). Lysis was limited to the periplasmic space by sonication at 4° C. with controlled pulses. Fabs in the periplasmic extracts were partially purified by ultracentrifugation at 12,000 rpm in a JA-10 rotor for 45 minutes at 4° C. Product was filtered and concentrated 10 times with Centricon filters.

The concentration of the partially purified Fabs was determined in the periplasmic extracts by sandwich ELISA using ImmunoPure Goat Anti-Human IgG [F(ab′)2] (Pierce; Cat. No. 31132), which was bound onto the surface of 96-well plate (Costar; Cat no. 3690). After a 1-hour incubation at 37° C., the plate was washed 6 times with deionized water and blocked using 170 μl/well PBS with 3% BSA. After a further 1-hour incubation at 37° C., 50 μl of a serial 3-fold dilution of each Fab, or known concentrations of a control human Fab (Cappel; Cat. No. 6001-0100), in PBS with 1% BSA were added to each well and incubated at 37° C. for 1 hour. The plate was then washed 6 times with TPBS (PBS with 0.05% Tween-20). The antibody binding was then determined by adding 50 μl of alkaline phosphatase conjugated goat anti-human antibody (Pierce; Cat. No. 31312) and incubated at 37° C. for 1 hour. Plate washing was repeated as above with TPBS and 100 μl disodium p-nitrophenyl phosphate (Sigma) was added to each well. ELISA reactions were developed for 60 minutes and the results were plotted against the control human Fab.

Results

A library of recombinant phage was generated according to the pDD technology (WO07/007,154) and panned on protein extracts obtained from a cell line of human fibroblasts infected with a clinical isolate of VZV. The five rounds of panning were also performed in parallel with the same library using a control protein extract from the same cell line not infected with VZV.

By the third round, the phage titer of the sample panned against the control protein extract was below 10⁴, meanwhile the phage titer of the sample panned against the VZV extract was more than 10⁴ at the third round, reaching 10⁵ at the fifth round. This value demonstrates the progressive enrichment of the library in recombinant phage expressing on their surface Fabs binding VZV antigens.

More than 260 clones obtained after the fifth round of panning were individually tested in ELISA and 86 of them were confirmed as positive. The PCR and sequence analysis of the HCDR3 in the selected clones identified two heavy chains characterizing the human Fabs now named DDF-VZV1 and DDF-VZV2. The reactivity of recombinant phage expressing DDF-VZV1 or DDF-VZV2 was tested against the VZV-specific protein extract as well as against unrelated antigens (uninfected cells, Bovine Serum Albumin), confirming their strong binding activity in ELISA format (FIG. 1). The specificity of the binding was also confirmed using Influenza virus antigens, for which the selected Fabs did not show any significant affinity.

The DNA sequences of the full heavy and light chains variable regions of these Fabs were determined, together with the corresponding CDRs, for DDF-VZV1 and DDF-VZV2 (FIGS. 2 and 3). These Fabs were recloned also into other vectors for obtaining sufficient recombinant protein for further assays, using E. coli-based systems for protein expression.

### Example 2

### Properties of DDF-VZV1 and DDF-VZV2 Tested on VZV-infected Cell Cultures

Materials & Methods

Neutralization Assay of VZV-Specific Human Fabs

The plaque-reduction assay was performed in Costar 24-well plates using 10⁴-10⁵ MRC-5 cells, inoculated into the plates under conditions where confluent monolayers usually form after 72 hours incubation at 37° C. VZV virus strain used was a clinical isolate.

The Fabs were partially purified as indicated in Example 1 and mixed at various concentrations (0.01, 0.1, 1, 10 and 50 μg/ml) with equal volumes of VZV cell-free stock suspended in maintenance medium (ELLEN strain; multiplicity of infection 0.01). The controls were constituted of equal volumes of maintenance medium and virus, in the absence of Fabs (blank control), or in the presence of an irrelevant Fab specific for human Hepatitis C virus (e137; Bugli F et al., 2001).

After 1 hour of incubation at 37° C., 250 μl of virus-fragment Fab mixtures or control mixtures were inoculated into wells (in duplicate) from which medium was removed. The plates were incubated for 2 hours at 37° C. to allow adsorption of unneutralized virus. The inocula were removed and 1.5 ml of maintenance medium was added. After 1 week of incubation in cell culture conditions, cells were washed with PBS, fixed with ethanol for 10 minutes at room temperature and stained with 1% crystal violet solution for 10 minutes. Plates were washed with distilled water three times and lysis plaques were counted. Neutralizing ability of each Fab was determined by counting single lysis plaques and calculating the percentage of reduction in viral plaque counts compared with the control samples.

Neutralizing ability of each Fab was determined by counting single fluorescing cells by fluorescence microscopy (Olympus), and calculating the percentage of reduction in number of VZV-positive cells compared with the control samples.

Immunofluorescence Analysis

MRC-5 cells were cultured in Costar 24-well plates containing sterile glass coverslips. When cell cultures were confluent as monolayers (usually form after 7 days incubation at 37° C.), the cells were infected with virus-Fab mixtures or control mixtures as follows. The medium was eliminated and cell monolayers were infected at high multiplicity of infection using VZV Ellen reference strain (250 μl/well; multiplicity of infection 0.1-1), pre-incubated with the indicated concentrations of semi-purified Fabs for 1 hour of incubation at 37° C. The controls were constituted of equal volumes of maintenance medium and virus, in the absence of Fabs (blank control), or in the presence of an irrelevant Fab specific for human Hepatitis C virus (e8). The plates were incubated for 2 hours at 37° C. to allow adsorption of unneutralized virus. The inocula were removed and 1.5 ml of MEM with 2% FBS was put in each well.

The Immunofluorescence Assay was performed 72 hours post-VZV W infection. After removing medium, cell monolayers were washed once with PBS and fixed in cold methanol-acetone solution (1:2 ratio; conserved at −20° C.) for 10 minutes at room temperature. Fixed cells were incubated with preparations of DDF-VZV1 or DDF-VZV2 as primary antibody for 30 minutes at 37° C. in a humid atmosphere, washed with PBS and finally incubated with anti-human IgG Fab specific FITC-Conjugate (Sigma) for 30 minutes at 37° C. in humid atmosphere. As controls, the cells were prepared using the secondary FITC-labeled antibody only, with a commercial anti-VZV antibody used according to manufacturer's instructions (Argene; Cat. No. 11-017). Additionally, MRC-5 cells infected with Cytomegalovirus were tested using the same preparations of DDF-VZV1 or of DDF-VZV2 as primary antibody. The slides were counterstained with Evans Blue, mounted with glycerol buffer and finally observed by a fluorescence microscope (Olympus).

Results

Further analyses of neutralization and binding activity for both DDF-VZV1 and DDF-VZV2 were performed by using preparations of partially purified Fabs.

In immunofluorescence, DDF-VZV1 stains more intensely VZV-infected cells in the (peri)-nuclear regions, where VZV virions are assembled, in comparison to a commercial murine monoclonal antibody that shows a nuclear staining in infected cells. DDF-VZV2 stains more intensely VZV-infected cells in the cytoplasmatic region (FIG. 4). No staining was obtained in absence of primary antibody or using DDF-VZV1 and DDF-VZV2 on MRC-5 cells not infected or infected with Cytomegalovirus. These data were also confirmed by immunofluorescence performed on cells obtained from the skin of individuals infected with VZV that have been briefly cultured and then observed in immunofluorescence.

The data on plaque reduction indicate that DDF-VZV1 and DDF-VZV2 are endowed with a strong neutralizing activity when preincubated with VZV. In fact, when compared to the controls (VZV without a Fab or with an unrelated Fab), the addition of these Fabs determines a reduction of plaque formation in a dose-dependent manner (FIG. 5).

### Example 3

### Production and Validation of DDF-VZV1 and DDF-VZV2 Using a pDD-Compatible Expression Vector

Materials & Methods

Design and Construction of pDLac-FLAGhis Vectors

A pDD vector containing the DDb cassette in which the Zeocine gene is used as marker gene (WO 07/007,154) has been modified by substituting the SpeI-NheI fragment including the cp3* sequence with a NheI-SpeI synthetic linker containing the two tags and a stop codon. Such linker was generated by annealing two oligonucleotides (SEQ ID NO.: 15 and 16). The resulting double-stranded DNA molecule was digested with SpeI and NheI and prepared for the cloning step into the corresponding linearized pDD vector. The final vector was characterised by restriction and sequence analysis in order to confirm the in frame insertion of the two tag sequences. The Lad gene was PCR-amplified from a commercially available plasmid named pET28 (Invitrogen) and was then inserted in the Stul site between the Zeocin gene and the LacZ promoter driving the expression of the protein to be fused to the FLAGhis tag.

PCR reactions were performed using a mix containing DNA template (20-30 ng), commercial PCR buffer (1×; Invitrogen), primers (0.2 μM), dNTP (0.25 mM), Taq DNA Polymerase (0.25 Units; Invitrogen), and water (to a final volume of 50 μl). The reaction was carried out in a Thermal Cycler (Perkin-Elmer) as follows: 5 minutes 94° C., 30 cycles of 30 seconds at 94° C., 30 seconds at 50-55° C., 1 minute at 72° C. The site directed mutagenesis PCR was performed in the same amplification conditions as above explained using the Pfu fusion Taq polymerase (Stratagene). The digestion of DNA with restriction enzyme was performed according to manufacturer's instructions (New England Biolabs). Bacteriophage T4-DNA ligase (Boehringer) was used for the ligation of the prepared DNA fragments into the appropriate vectors. The molar ratio of vector:insert DNA was 1:3 and the total concentration of DNA was approximately 50 ng. The ligation reaction was performed in 20 μls as follows: DNA (50 ng), Ligation buffer (1×), 10 mM ATP (1 μl), T4 DNA ligase (4 Units), and water (up to 20 μl). The T4 ligation reaction was carried out overnight at 15° C. TG1 or XL-1 Blue Escherichia coli competent cells were prepared using CaCl₂ protocols and transformed with the ligation mixtures including pDLac-FLAGhis vectors.

The sequence coding the heavy and light chains of DDF-VZV1 and DDF-VZV2 were cloned in pDLac-FLAGhis using the appropriate restriction sites. The final vectors (pDLac-VZV1-FLAGhis and pDLac-VZV2-FLAGhis) were characterised by restriction and sequence analysis in order to confirm the in frame insertion of the two antibody sequences.

Expression, Purification, and Detection of FLAGhis-Tagged DDF-VZV1 and DDF-VZV2

E. coli XL-1 Blue were transformed pDLac-VZV1-FLAGhis, pDLac-VZV2-FLAGhis, or a pDLac-FLAGhis variant expressing e509, an HCV-specific Fab (Bugli et al., 2001) to be used as control.

The resulting strains were used for the production of Fabs in bacterial cultures. For this purpose a single colony obtained from transformation was inoculated into 10 mls of SB medium containing tetracycline (10 μg/ml), ampicillin (100 μg/ml) and zeocin (50 μg/ml). The bacteria were let grown for 12 hours at 37° C. After the incubation time, 500 μl of them were diluted in 100 mL of SB supplied of the same antibiotics and let grown for 6-8 hours until they reached 0.6 OD. Isopropyl-beta-D-thio-galactopyranoside (IPTG) was added to a final concentration of 1 mM and cells were incubated for additional 14 hours at 30° C. in a rotatory shaker. Bacteria were harvested by centrifugation at 5000 g for 20 minutes, resuspended in 1 mL of phosphate buffered saline (PBS) and lysed by a freeze-thawing procedure (three steps at −80° C. and 37° C.). Cell debris was removed by centrifugation in a microfuge at 15,000 g for 5 minutes at room temperature.

The supernatants were used for Western Blot analysis with anti-Fab and anti-His antibodies detection in order to check the Fab expression. SDS-PAGE was made loading the samples with or without β-mercaptoethanol (reducing and non-reducing conditions), then blotting was performed for 2 hours at 350 mA; nitrocellulose paper was first stained with Ponceaus Red to verify the occurred blotting and than it was let in agitation overnight at 4° C. with 10% milk/PBS/Tween 0.1% (blocking solution). After one wash with PBS/Tween 0.1%, the nitrocellulose was incubated in agitation with horseradish peroxidase (HRP)-conjugated anti-Fab antibody (Sigma) or with HRP-conjugated anti-His-tag antibody (Roche) at room temperature for 1 hour. After three times washing with PBS/Tween 0.1%, SuperSignal West Pico Chemiluminescent substrate (Pierce) was added and the films was developed for 30 seconds, 1 and 5 minutes respectively.

The signal detected with anti-Fab was compared with the anti-His HRP-conjugated detection. Both of them revealed a major band of ˜60 kDa corresponding to the native form of Fab fragment (when the clarified cell extract was run in a gel using non-reducing conditions). When the same sample was subject to a SDS-PAGE using reducing conditions, only the heavy chain (˜25 kDa) was if using the anti-His antibody. The detection with the anti-Fab revealed the presence of the light chain that could not be detected because was not tagged.

For a larger scale production of the FLAGhis-tagged fabs, one litre-aliquot of super broth containing tetracycline (10 μg/mL), ampicillin (100 μg/mL) and zeocin (50 μg/ml) was inoculated with one bacterial colony transformed with the specific pDLac-FLAGhis vector, and grown at 37° C. for 7 h in a rotatory shaker, induced with IPTG as indicated and grown overnight at 30° C. Cells were harvested by centrifugation, resuspended with 25 mL of PBS and sonicated. Cell debris was eliminated by centrifugation (15,000 g for 50 minutes), and 0.22 μm filtered supernatant was purified by IMAC. A standard Qiagen purification protocol was followed: NiNTA resin (Qiagen) was equilibrate with Binding Buffer solution (Phosphate buffer, 10 mM Imidazole) and then the filtered supernatant was mixed with the resin (300 μL resin/100 mL colture) and left in agitation for 1 hour at 4° C. This solution was centrifuged at 400 g for 5 minutes to recover the Fab fragment linked to the resin and the flow through was collected. The resin was washed with 5 volumes of Binding Buffer 10 mM imidazole to eliminate the aspecific binding. The His-Flag Fab fragment was eluted with 3 volume of Elution Buffer (Phosphate buffer, 500 mM NaCl, 500 mM imidazole). Eluted aliquots were analysed by SDS/12.5% polyacrylamide gel electrophoresis and detected with Coomassie brilliant-blue stain and Western Blot.

Results

The activity of DDF-VZV1 and DDF-VZV2 has been initially tested as partially purified Fabs and recombinant phage. Further validation requires to test such antibody fragments in the form of purified recombinant proteins using an appropriate bacterial expression system.

Phagemids carrying the Fab of interest are usually transferred into suitable expression vectors and/or appropriate bacterial strain (e.g. amber non-suppressor) for the production of soluble molecules. At the scope of avoiding such time-consuming steps of molecular cloning, a more effective approach would be to generate an expression vector that has restriction sites compatible with those in original pDD vectors that were used for identifying such Fabs. Moreover, the presence of a tag sequence would allow easier purification and detection of the Fab.

Such expression vector, named pDLac-FLAGhis was designed and constructed. It is based on a pDD vector in which the coding sequence for cp3* has been substituted with a sequence coding for two protein tags (a FLAG peptide, containing 8 amino acids, and 6-Histidine tag) followed by stop codon (FIG. 6A), The Histidine tag allows the Fab to be purified by affinity chromatography, using for example the IMAC (Immobilization Metal Affinity Chromatography) purification system. The FLAG tag may be very useful in detection assay (such as ELISA). Vectors in which these two tags has been cloned and fused to recombinant proteins, as well means for detecting or purifying the FLAGhis-tagged proteins have been described in the literature (Pagny S et al., 2000; Koivunen P et al., 2004; Komatsu M et al., 2004).

A Fab (or any other protein sequence) that has been initially identified and characterized using a pDD phagemid can be extracted using the DD cassette which is then cloned directionally in a pDLac-FLAGhis vector. In this way the sequence originally fused to a coat protein is now cloned in frame with the FLAG tag and poly-Histidine tag at the C-terminus. The resulting vector contains the original marker gene within the DD cassette (e.g the Zeocin gene), allowing the selection of the vector containing the desired insert. As a mean for controlling the transcription of the proteins to be expressed using a LacZ promoter within a pDLac-FLAGhis vector, a LacI gene has been inserted in the original DD cassette (FIG. 6B).

This cloning strategy provides two advantages. A first one is to increase the space between the two LacZ promoter regions in the original DD cassette avoiding possible homologous recombination. The second is the repressor protein codified from the Lad gene, known to be able to down regulate the expression system blocking with its binding to the operator region the LacZ promoter. Induction is then modulated using appropriate concentrations of IPTG and/or glucose. Upon induction, both the sequence cloned in the under the control of the LacZ promoter/PelB signal sequence are expressed and targeted to the periplasm, where the pelB sequence is subsequently cleaved by the enzyme signal peptidase. Within the periplasm, appropriate oxidizing conditions allow for the formation of the disulfide bonds in the recombinant proteins and the correct protein folding (for example the light and heavy chains of a Fab that are assembled into a heterodimeric protein). The FLAGhis-tagged protein can be purified to homogeneity by eluting and testing fraction of a column for affinity chromatography (FIG. 6B). Then, this protein can be identified in Western blot or immunofluorescence using, for example, a commercial anti-FLAG monoclonal antibody (available from Sigma).

Using this approach, FLAGhis-tagged version of DDF-VZV1 and DDF-VZV2 (FIG. 7), as well as of a control fab, were produced and tested in assays described above. These recombinant variants of the selected Fabs confirmed the original observations regarding the VZV-neutralizing activity, showing an IC50 around 2 μg/ml (FIG. 8A). However, when the two Fabs are combined in a single preparation, the VZV-neutralizing activity of this preparation resulted superior of a preparation containing the identical amount of a single Fab. In fact, if a preparation containing a single Fab increases its activity of 10-15% when the Fab concentration goes from 1 to 2 μg/ml, a preparation containing 1 μg/ml of DDF-VZV1 and 1 μg/ml of DDF-VZV1 has a much higher VZV-neutralizing activity with an IC50 value below 2 μg/ml (FIG. 8B). This effect, possibly due to the different VZV-specific epitope recognized by the two Fabs (see FIG. 4), suggests that pharmaceutical compositions containing DDF-VZV1 and DDF-VZV2 (or two antibodies or antibody fragment having similar properties) may provide a better therapeutic or prophylactic effect against VZV infection and VZV-related diseases.

The experimental evidence presented here makes DDF-VZV1 and DDF-VZV2 (or alternative protein sequences based on their specific HCDR3s and showing similar properties) candidate compounds for diagnostic, therapeutic, or prophylactic applications related to VZV infection and VZV-related diseases.

### REFERENCES

Abdel-Haq N et al., (2006). Indian J. Pediatr. 73: 313-21.
Aldrich T et al., (2003). Biotechnol Prog. 19: 1433-8.
Andrei G et al., (2005a). Antimicrob Agents Chemother. 49: 4671-80.
Andrei G et al., (2005b). Antimicrob Agents Chemother. 49: 1081-6.
Arvin A (1996). Clin Microbiol Rev. 9: 361-81.
Baiker A et al., (2004). Proc Natl Acad Sci USA. 101: 10792-7.
Barrios Y et al., (2004). J Mol. Recognit. 17:332-8.
Bayry J et al., (2007). Nat Clin Pract Neurology. 3:120-1.
Benhar I, (2001). Biotechol Adv. 19: 1-33.
Berarducci B et al., (2006). J Virol 80: 9481-96.
Bianchi A and McGrew J, (2003). Biotechnol Bioeng. 84: 439-44.
Bohm E et al., (2004). Biotechnol Bioeng. 88: 699-706.
Bond C et al., (2003). J Mol. Biol. 332: 643-55.
Bradbury A and Marks J (2004). J Immunol Methods. 290: 29-49.
Brereton H et al., (2005). Br J. Opthalmol. 89: 1205-9.
Bugli F et al., (2001). J. Virol., 75: 9986-9990.
Burgoon M et al., (2003). Ann Neurol. 54: 459-63.
Burioni R et al., (1998). Hepatology. 28: 810-4.
Burioni R et al., (2001). Human Antibodies. 10: 149-154.
Butler M, (2005). Appl Microbiol Biotechnol. 68: 283-91.
Carby M et al., (2007). J Heart Lung Transplant. 26: 399-402.
Carton J et al., (2007). Protein Expr Purif. 55: 279-86.
CDC, (2006). MMWR Morb Mortal Wkly Rep. 55: 209-10.
Chapman A et al., (1999). Nat. Biotechnol. 17: 780-3.
Chatenoud L, Methods Mol. Med. 2005. 109: 297-328.
Chaves S et al., (2007). N Engl J. Med. 356: 1121-9.
Chaves Tdo S. et al., (2005). Pediatr Transplant. 9: 192-6.
Chen J et al., (2004). Cell. 119: 915-26.
Conrad U and Scheller J, (2005). Comb Chem High Thr Screen. 8: 117-26.
Corisdeo S and Wang B, (2004) Protein Expr Purif. 34: 270-9.
Dinnis D and James D, (2005). Biotechnol Bioeng. 91: 180-9.
Drew P et al., (2001). J Gen Virol. 82: 1959-63.
Dunman P and Nesin M, (2003). Curr Opin Pharmacol. 3: 486-96.
Dworkin R et al., (2007). Clin. Infect Dis. 44: S1-26.
Fang J et al., (2005). Nat. Biotechnol. 23: 584-90.
Finnen R et al., (2006). J. Virol. 80: 10325-34.
Forghani B et al., (1994). Virology, 199: 458-62.
Foung S et al., (1985). J Infect Dis. 152: 280-5.
Fowler W et al., (1995). Virology. 214: 531-40.
Furebring C et al., (2002). Mol. Immunol. 38: 833-40.
Garcia-Valcarcel M et al., (1997). Vaccine. 15: 709-19.
Gilden D, (2004). Herpes. 11 Suppl 2: 89A-94A.
Gilliland L et al., (1996). Tissue Antigens. 47: 1-20.
Giudicelli V et al., (2004). Nucl. Acids Res. 32: W435-440.
Grose C et al., (1983). Infect Immun. 40: 381-8.
Grose C, (2006). Herpes. 13: 32-6.
Grote A et al., (2005). Nucleic Acid Res. 33: W526-31.
Grunberg J et al., (2003). Biotechniques. 34: 968-72.
Guttieri M et al., (2003). Hybrid Hybridomics. 22: 135-45.
Hackanson B et al., (2005). Clin Transplant 19: 566-70.
Hambleton S and Gershon A, (2005). Clin Microbiol Rev. 18: 70-80.
Hambleton S et al., (2007). J. Virol. 81: 7548-58.
Harris R et al., (2004). Drug Develop Res. 61: 137-154.
Hasnie F et al., (2007). Neuroscience. 144: 1495-508.
Haumont M et al., (1997). J Med. Virol. 53, 63-8.
Hood C et al., (2003). J. Virol. 77: 12852-64.
Horenstein A et al., (2003). J Immunol Methods. 275: 99-112.
Huang Y et al., (2001). Eur J. Pediatr. 160: 91-4.
Huse K et al., (2002). J Biochem Biophys Methods. 51: 217-31.
Hust M and Dubel S, (2005). Methods Mol. Biol. 295: 71-96.
Ito M et al., (1993). J Infect Dis. 168: 1256-9.
Jain M et al., (2007). Trends Biotechnol. 25: 307-16.
Jensen L et al., (2004). J Immunol Methods. 284: 45-54.
Kausmally L et al., (2004). J Gen Virol. 85: 3493-500.
Keller M and Stiehm E, (2000). Clin Microbiol Rev. 13: 602-14.
Kezuka T, (2004). Ocul Immunol Inflamm. 12: 17-24.
Kim S et al., (2005). Mol. Cells. 20: 17-29.
Kiss C et al., (2006). Nucleic Acids Res. 34: e132.
Kjartansdottir A et al., (1996). Arch Virol. 141: 2465-9.
Knappik A et al., (2000). J Mol. Biol. 296: 57-86.
Koivunen P et al., (2004). J. Biol. Chem. 279: 9899-9904.
Komatsu Metal., (2004). EMBO J. 23: 1977-1986.
Koren G et al., (2002). J Clin Pharmacol. 42: 267-74.
Krah D, (1996). Infect Dis Control North Amer. 10: 507-527.
Ku C et al., (2005). J. Virol. 79: 2651-8.
Kutinova L et al., (2001). Virology. 280: 211-20.
Ladner R, (2007). Nature Biotechnol. 25: 875-77.
Laffly E and Sodoyer R, (2005). Hum. Antibodies. 14: 33-55.
Levi M et al., (2000). AIDS Res Hum Retroviruses. 16: 59-65.
Li Q et al., (2007). J. Virol. 81: 8525-32.
Liesegang T, (2004). Curr Opin Ophtahal. 15: 521-6.
Lloyd-Evans P and Gilmour J, (2000). Hybridoma. 19: 143-9.
Lobo E et al., (2004). J Pharm Sci. 93: 2645-68.
Logtenberg T, (2007). Trends Biotechnol. 25: 390-4.
Ludwig B. et al., (2006). Infection. 34: 222-6.
Ma J et al., (2005). Vaccine. 23: 1814-8.
Mancini N et al., (2004). New Microbiol. 27: 315-28.
Mantis N et al., (2007). J. Immunol. 179: 3144-52.
Maple P et al., (2006). Clin Vaccine Immunol. 13: 214-8.
Massaer M et al., (1999). Viral Immunol. 12: 227-36.
Matsuo K et al., (2003). J. Dermatol. 30: 109-15.
Montalvo E and Grose C (1986). Virology. 149: 230-41.
Mori I and Nishiyama Y, (2005). Rev Med. Virol. 15: 393-406.
Murray A et al., (2002). J Chromatogr Sci. 40: 343-9.
Nardone A et al., (2007). Vaccine. 25: 7866-72.
Nemeckova S et al., (1996). J Gen Virol. 77: 211-5.
Nilsson J et al., (1997). Protein Expr Purif. 11: 1-16.
Nisnevitch M and Firer M, (2001). J Biochem Biophys Methods. 49: 467-80.
Norberg P et al., (2006). J. Virol. 80: 9569-76.
Nwanegbo E et al., (2007) Invest Opthalmol V is Sci. 48: 4171-6.
Ohlin M et al., (1993). J. Virol. 67: 703-10.
Oxman M et al., (2006). N Engl J. Med. 352: 2271-84.
Pagny S et al., (2000). Plant Cell. 12: 739-756.
Persic L et al., (1997). Gene. 187: 9-18.
Quinlivan M and Breuer J, (2006). Rev Med. Virol. 16: 225-50.
Quinlivan M et al., (2007). Proc Natl Acad Sci USA. 104: 208-12.
Rodi D et al., (2002). J Mol. Biol. 322: 1039-52.
Rojas G et al., (2004). J Immunol Methods. 293: 71-83.
Roque A et al., (2004). Biotechnol Prog. 20: 639-5.
Sandherr M et al., (2006). Ann Oncol. 17: 1051-9.
Sauerbrei A and Wutzler P, (2006). J Clin Microbiol. 44: 3094-7.
Sauerbrei A and Wutzler P, (2007). Med Microbiol Immunol. 196: 95-102.
Schlatter S et al., (2005). Biotechnol Prog. 21: 122-33.
Schleiss M, (2003). Herpes. 10: 4-11.
Shankar V et al., (2005). Hybridoma (Larchmt). 24: 50-4.
Smith J et al., (2001). Clin Diagn Lab Immunol. 8: 871-9.
Sorensen H and Mortensen K, (2005). J. Biotech. 115: 113-28.
Sugano T et al., (1987). Eur J. Immunol. 17: 359-64.
Suzuki K et al., (2007). J Med. Virol. 79: 852-862.
Tabrizi M and Riskos L, (2007). Drug Disc Today. 12: 540-7.
Talukder Y et al., (2005). J Virol Methods. 128: 162-7.
Taylor S and Moffat J, (2005). J. Virol. 79: 11501-6.
Tyring S, (2007). J Am Acad Dermatol. 57(6 Supply: S136-42.
Vafai A and Yang W, (1991). J. Virol. 65: 5593-6.
Venturi Metal., (2002). J Mol. Biol. 315:1-8.
Verdoliva A et al., (2002). J Immunol Methods. 271: 77-8.
Weinberg J, (2007). J Am Acad Dermatol. 57(6 Suppl): S130-5.
Wijkhuisen A et al., (2003). Eur J. Pharmacol. 468: 175-82.
Williamson R et al., (1993). Proc Natl Acad Sci USA. 90: 4141-5.
Wu L and Forghani B, (1997). Arch Virol. 142: 349-62.
Xu J and Davis M, (2000). Immunity. 13: 37-45.
Yokoyama T et al., (2001). J Gen Virol. 82: 331-4.
Yoon S et al., (2004). Biotechnol Prog. 20: 1683-8.
Zerboni L et al., (2005). Proc Natl Acad Sci USA. 102: 6490-5.

1. A protein comprising a sequence having at least 90% identity with SEQ ID NO.: 5.

2. The protein according to claim 1, wherein said protein comprises a sequence having at least 90% identity with SEQ ID NO.: 2.

3. The protein of claim 1, wherein said protein further comprises a sequence having at least 90% identity with SEQ ID NO.: 7.

4. The protein of claim 1, wherein said protein is an antibody, an antibody fragment, or a fusion protein.

5. The protein of claims claim 4, wherein said antibody fragment is a variable heavy/light chain heterodimer, or a single-chain fragment variable.

6. The protein of claim 1 wherein such protein binds and neutralizes Varicella Zoster virus (VZV).

7. A nucleic acid molecule encoding a protein of claim 1.

8. The nucleic acid molecule of claim 7, wherein said nucleic acid has at least 90% identity with of SEQ ID NO.: 1.

9. A vector comprising a nucleic acid of claim 7.

10. A recombinant phage, a prokaryotic host cell, or an eukaryotic host cell comprising a nucleic acid of claim 7.

11. (canceled)

12. (canceled)

13. A therapeutic, prophylactic, or diagnostic composition comprising a protein of claim 1.

14. The composition of claim 13 wherein the composition is for ocular or topical administration.

15. The composition of claim 13, further comprising a different VZV-neutralizing antibody, VZV-neutralizing antibody fragment, an intravenous immunoglobulins preparation, a steroid, and/or an antiviral compound.

16. A method of producing a protein of claim 1 using a nucleic acid of claim 7.

17. A method of producing a protein of claim 1 using a recombinant phage or a host cell of claim 10.

18. A method of detecting, treating, inhibiting, preventing, and/or ameliorating VZV infection and/or re-infection using a protein of claim 1.


SEQUENCE LISTING (26 sequences)

SEQ ID NO: 1 - DNA, 384 bp, Homo sapiens
ctcgagtctggcccaggactggtgaagccttcggagaccctgtccctcacctgctctgtctctggtggctccatcaccactaaagacttctcctgggcttggatccgccagcccccagggaaggggctggagtggattgggagtatctattatagtgggaccacccactacagcccgtccctcaagagtcgagtcatcatgtccgtggacacgtccgacaataagttttccctgagtctgacctctgtgaccgccgcagacacggctgtttattactgtgcgagacaactggggagaagtccagggatttacagtttttggagagatcatagaacctctgcttttgacttctggggccagggaaccctggccaccgtctccgca

SEQ ID NO: 2 - protein, 128 aa, Homo sapiens
LESGPGLVKPSETLSLTCSVSGGSITTKDFSWAWIRQPPGKGLEWIGSIYYSGTTHYSPSLKSRVIMSVDTSDNKFSLSLTSVTAADTAVYYCARQLGRSPGIYSFWRDHRTSAFDFWGQGTLATVSA

SEQ ID NO: 3 - protein, 10 aa, Homo sapiens
GGSITTKDFS

SEQ ID NO: 4 - protein, 7 aa, Homo sapiens
IYYSGTT

SEQ ID NO: 5 - protein, 24 aa, Homo sapiens
ARQLGRSPGIYSFWRDHRTSAFDF

SEQ ID NO: 6 - DNA, 318 bp, Homo sapiens
gagctcacgcagtctccatcctccctgtctgcatctgtaggagacagagtcaccatcacttgccgggcaactcagagcattagtagttatttaagttggtatcagcagaaaccagggcaagcccctaagctcctgatctatggtgcatccaatttgcaaagtggggtcccatcaaggttcagtggcagtggatctgggacagatttcactctcacgatcaacagtctgcagcctgaagattttgcaacttactactgtcaacagactctgagtacccctttcactttcggccctgggaccagagtggatttcaaacga

SEQ ID NO: 7 - protein, 106 aa, Homo sapiens
ELTQSPSSLSASVGDRVTITCRATQSISSYLSWYQQKPGQAPKLLIYGASNLQSGVPSRFSGSGSGTDFTLTINSLQPEDFATYYCQQTLSTPFTFGPGTRVDFKR

SEQ ID NO: 8 - DNA, 375 bp, Homo sapiens
ctcgagtcggggggaggcttggtacagcctggggggtccctgagactctcctgtgaagcctcaggattcaccttcagttcctacaccatgaactgggtccgccaggctccagggaaggggctggagtgggtttcatggattagtagtagtggtagtgacatatattacgcagaatctgtgaagggccgattcaccatctccagagacaatgccaagaactcactgtacctgcaaatgaacagcctgagagacgaggacacggctctttattactgtgcgagagatggaattgacagcagcagcagctgggggggagagccgtacttctacactatggacttctggggccaagggaccacggtcatcgtctcctca

SEQ ID NO: 9 - protein, 125 aa, Homo sapiens
LESGGGLVQPGGSLRLSCEASGFTFSSYTMNWVRQAPGKGLEWVSWISSSGSDIYYAESVKGRFTISRDNAKNSLYLQMNSLRDEDTALYYCARDGIDSSSSWGGEPYFYTMDFWGQGTTVIVSS

SEQ ID NO: 10 - protein, 8 aa, Homo sapiens
GFTFSSYT

SEQ ID NO: 11 - protein, 8 aa, Homo sapiens
ISSSGSDI

SEQ ID NO: 12 - protein, 22 aa, Homo sapiens
ARDGIDSSSSWGGEPYFYTMDF

SEQ ID NO: 13 - DNA, 336 bp, Homo sapiens
gagctcactcagtctccagactccctggctgtgtctctgggcgagagggccaccatcaactgcaagtccagccagagtgttttatactactccaacaagaagaactacttagcttggtaccagcagaaaccaggacagcctcctaagctactaatttactgggcatccacccgggaatccggggtccctgaccgattcagtggcagcgggtctgggacagatttcactctcaccatcagcagcctgcaggctgaagatgtggcagtttattactgtcagcaatattatagtactcctctcactttcggcggagggaccaaggtggagatcaaacga

SEQ ID NO: 14 - protein, 112 aa, Homo sapiens
ELTQSPDSLAVSLGERATINCKSSQSVLYYSNKKNYLAWYQQKPGQPPKLLIYWASTRESGVPDRFSGSGSGTDFTLTISSLQAEDVAVYYCQQYYSTPLTFGGGTKVEIKR

SEQ ID NO: 15 - DNA, 75 bp, Artificial
  FLAGhis-Forward oligonucleotide
gacaaaactagtgactacaaggacgatgacgataagcatcaccatcaccatcattgagctagcgaattctagtaa

SEQ ID NO: 16 - DNA, 75 bp, Artificial
  FLAGhis-Reverse oligonucleotide
ttactagaattcgctagctcaatgatggtgatggtgatgcttatcgtcatcgtccttgtagtcactagttttgtc

SEQ ID NO: 17 - protein, 14 aa, Artificial
  FLAGhis tag protein sequence
DYKDDDDKHHHHHH

SEQ ID NO: 18 - DNA, 3735 bp, Artificial
  Fragment of pDLacI-VZV1tag vector
gctagctcaatgatggtgatggtgatgcttatcgtcatcgtccttgtagtcactagctcaatgatggtgatggtgatgcttatcgtcatcgtccttgtagtcactagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgcggagacggtggccagggttccctggccccagaagtcaaaagcagaggttctatgatctctccaaaaactgtaaatccctggacttctccccagttgtctcgcacagtaataaacagccgtgtctgcggcggtcacagaggtcagactcagggaaaacttattgtcggacgtgtccacggacatgatgactcgactcttgagggacgggctgtagtgggtggtcccactataatagatactcccaatccactccagccccttccctgggggctggcggatccaagcccaggagaagtctttagtggtgatggagccaccagagacagagcaggtgagggacagggtctccgaaggcttcaccagtcctgggccagactcgagcagtttcacctgggccatggctggttgggcagcgagtaataacaatccagcggctgccgtaggcaataggtatttcattatgactgtctccttaagctagtttagaatttgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgaattcctgcagcccgatccaagctcccgggagctttttgcaaaagcctaggcctccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaggccttgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtggggacgggagttcgccctgcgcgacccggccggcaactgcgtgcacttcgtggccgaggagcaggactgacactcgacctcgaaacttgtttattgcagcttataatggttacaaataaagcaatagcatcacaaatttcacaaataaagcatttttttcactgcattctagttgtggtttgtccaaactcatcaatgtatcttatcatgtctagaattaacactctcccctgttgaagctctttgtgacggggctactcaggccctgatgggtgacttcgcaggcgtagactttgtgcttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagttcgtttgaaatccactctggtcccagggccgaaagtgaaaggggtactcagagtctgttgacagtagtaagttgcaaaatcttcaggctgcagactgttgatcgtgagagtgaaatctgtcccagatccactgccactgaaccttgatgggaccccactttgcaaattggatgcaccatagatcaggagcttaggggcttgccctggtttctgctgataccaacttaaataactactaatgctctgagttgcccggcaagtgatggtgactctgtctcctacagatgcagacagggaggatggagactgcgtgagctcggccatggctggttgggcagcgagtaataacaatccagcggctgccgtaggcaataggtatttcattatgactgtctccttaagctagtttagaatttgtgtgaaattgttatccgctcacaattccacacaacatacgagccggaagcataaagtgtaaagcctggggtgcctaatgagtgagctaactcacattaattgcgttgcgcggccaggccggc

SEQ ID NO: 19 - DNA, 825 bp, Artificial
  DDF-VZV1 CH FLAGhis tagged sequence
atgaaatacctattgcctacggcagccgctggattgttattactcgctgcccaaccagccatggcccaggtgaaactgctcgagtctggcccaggactggtgaagccttcggagaccctgtccctcacctgctctgtctctggtggctccatcaccactaaagacttctcctgggcttggatccgccagcccccagggaaggggctggagtggattgggagtatctattatagtgggaccacccactacagcccgtccctcaagagtcgagtcatcatgtccgtggacacgtccgacaataagttttccctgagtctgacctctgtgaccgccgcagacacggctgtttattactgtgcgagacaactggggagaagtccagggatttacagtttttggagagatcatagaacctctgcttttgacttctggggccagggaaccctggccaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtgactacaaggacgatgacgataagcatcaccatcaccatcat

SEQ ID NO: 20 - protein, 275 aa, Artificial
  DDF-VZV1 CH FLAGhis tagged sequence
MKYLLPTAAAGLLLLAAQPAMAQVKLLESGPGLVKPSETLSLTCSVSGGSITTKDFSWAWIRQPPGKGLEWIGSIYYSGTTHYSPSLKSRVIMSVDTSDNKFSLSLTSVTAADTAVYYCARQLGRSPGIYSFWRDHRTSAFDFWGQGTLATVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSDYKDDDDKHHHHHH

SEQ ID NO: 21 - DNA, 702 bp, Artificial
  DDF-VZV1 CL recombinant sequence
atgaaatacctattgcctacggcagccgctggattgttattactcgctgcccaaccagccatggccgagctcacgcagtctccatcctccctgtctgcatctgtaggagacagagtcaccatcacttgccgggcaactcagagcattagtagttatttaagttggtatcagcagaaaccagggcaagcccctaagctcctgatctatggtgcatccaatttgcaaagtggggtcccatcaaggttcagtggcagtggatctgggacagatttcactctcacgatcaacagtctgcagcctgaagattttgcaacttactactgtcaacagactctgagtacccctttcactttcggccctgggaccagagtggatttcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaagcacaaagtctacgcctgcgaagtcacccatcagggcctgagtagccccgtcacaaagagcttcaacaggggagagtgt

SEQ ID NO: 22 - protein, 234 aa, Artificial
  DDF-VZV1 CL recombinant sequence
MKYLLPTAAAGLLLLAAQPAMAELTQSPSSLSASVGDRVTITCRATQSISSYLSWYQQKPGQAPKLLIYGASNLQSGVPSRFSGSGSGTDFTLTINSLQPEDFATYYCQQTLSTPFTFGPGTRVDFKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 816 bp, Artificial
  DDF-VZV2 CH FLAGhis tagged sequence
atgaaatacctattgcctacggcagccgctggattgttattactcgctgcccaaccagccatggcccaggtgaaactgctcgagtcggggggaggcttggtacagcctggggggtccctgagactctcctgtgaagcctcaggattcaccttcagttcctacaccatgaactgggtccgccaggctccagggaaggggctggagtgggtttcatggattagtagtagtggtagtgacatatattacgcagaatctgtgaagggccgattcaccatctccagagacaatgccaagaactcactgtacctgcaaatgaacagcctgagagacgaggacacggctctttattactgtgcgagagatggaattgacagcagcagcagctgggggggagagccgtacttctacactatggacttctggggccaagggaccacggtcatcgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtgactacaaggacgatgacgataagcatcaccatcaccatcat

SEQ ID NO: 24 - protein, 272 aa, Artificial
  DDF-VZV2 CH FLAGhis tagged sequence
MKYLLPTAAAGLLLLAAQPAMAQVKLLESGGGLVQPGGSLRLSCEASGFTFSSYTMNWVRQAPGKGLEWVSWISSSGSDIYYAESVKGRFTISRDNAKNSLYLQMNSLRDEDTALYYCARDGIDSSSSWGGEPYFYTMDFWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSDYKDDDDKHHHHHH

SEQ ID NO: 25 - DNA, 720 bp, Artificial
  DDF-VZV2 CL recombinant sequence
atgaaatacctattgcctacggcagccgctggattgttattactcgctgcccaaccagccatggccgagctcactcagtctccagactccctggctgtgtctctgggcgagagggccaccatcaactgcaagtccagccagagtgttttatactactccaacaagaagaactacttagcttggtaccagcagaaaccaggacagcctcctaagctactaatttactgggcatccacccgggaatccggggtccctgaccgattcagtggcagcgggtctgggacagatttcactctcaccatcagcagcctgcaggctgaagatgtggcagtttattactgtcagcaatattatagtactcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaagcacaaagtctacgcctgcgaagtcacccatcagggcctgagtagccccgtcacaaagagcttcaacaggggagagtgt

SEQ ID NO: 26 - protein, 240 aa, Artificial
  DDF-VZV2 CL recombinant sequence
MKYLLPTAAAGLLLLAAQPAMAELTQSPDSLAVSLGERATINCKSSQSVLYYSNKKNYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC